(19) United States
(12) Patent Application Publication
(10) Pub. No.: US 2021/0022613 A1
(43) Pub. Date: Jan. 28, 2021

(54) Wearable Device for Optical Breast Imaging and Spectroscopic Tissue Analysis

(71) Applicant: Robert A. Connor, St. Paul, MN (US)

(72) Inventor: Robert A. Connor, St. Paul, MN (US)

(73) Assignee: Holovisions LLC, St. Paul, MN (US)

(21) Appl. No.: 16/933,138

(22) Filed: Jul. 20, 2020

Related U.S. Application Data
(60) Provisional application No. 62/879,485, filed on Jul. 28, 2019.

(51) Int. Cl. A61B 5/00; A41C 3/00

(52) U.S. Cl. CPC A61B 5/0091; A41C 3/0064; A61B 5/6804; A61B 5/0075

(57) ABSTRACT

This invention is a wearable optical breast imaging device (such as a “smart bra”) with at least one cup, multiple light emitters on the interior of the cup which emit light beams at different times, and multiple light receivers on the interior of the cup. Changes in the spectra of light energy caused by transmission of the light energy through breast tissue are analyzed to diagnose whether there is abnormal tissue within the breast and, if so, to identify the location of abnormal tissue within the breast.

### CROSS-REFERENCE TO RELATED APPLICATIONS

This patent application claims the priority benefit of U.S. provisional patent application 62/879,485 entitled “ScanBra™ Wearable Optical Breast Imaging Device” filed on 2019 Jul. 28. The entire contents of this related application are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable medical devices for imaging and diagnosis.

### INTRODUCTION

Breast cancer is the most common form of cancer in women and a leading cause of death. Breast imaging can serve a critical role in the early diagnosis and treatment of breast cancer. However, there are limitations to frequent use of the current breast imaging modalities. For example, x-ray mammography is useful and commonly used, but long-term exposure to x-rays can promote cancer. Also, x-ray mammography has relatively low sensitivity. Magnetic Resonance (MR) breast imaging has relatively high sensitivity, but tends to have examination times that are relatively long for use in frequent screening. Also, MR imaging tends to be costly and have relatively low specificity. There remains a need for a new breast imaging modality which can be used frequently and safely for breast imaging and tissue analysis.

During recent years, there has been increased investigation of the possibilities of optical breast imaging using safe non-ionizing radiation such as visible, ultraviolet, infrared, and near-infrared light energy. However, thus far there have been limitations to optical breast imaging. For example, with current stationary optical imaging devices, it can be difficult to get close optical communication between the breast and the light emitters and receivers of the device due to differences in the sizes and shapes of breasts. With current handheld optical imaging devices, it can be difficult to accurately measure absolute tissue locations to track changes over time and to get a comprehensive image of the complete breast.

### Review of the Relevant Art

U.S. Pat. No. 5,876,339 (Lemire, Mar. 2, 1999, “Apparatus for Optical Breast Imaging”) discloses an optical breast imager with an adjustable volume which encloses a patient's breast. U.S. Pat. No. 5,999,836 (Nelson et al., Dec. 7, 1999, “Enhanced High Resolution Breast Imaging Device and Method Utilizing Non-Ionizing Radiation of Narrow Spectral Bandwidth”) and U.S. Pat. No. 6,345,194 (Nelson et al., Feb. 5, 2002, “Enhanced High Resolution Breast Imaging Device and Method Utilizing Non-Ionizing Radiation of Narrow Spectral Bandwidth”) disclose breast imaging using collimated non-ionizing radiation in the near ultraviolet, visible, infrared, and microwave regions.

U.S. Pat. No. 6,240,309 (Yamashita et al., May 29, 2001, “Optical Measurement Instrument for Living Body”), U.S. Pat. No. 6,640,133 (Yamashita et al., Oct. 28, 2003, “Optical Measurement Instrument for Living Body”), and U.S. Pat. No. 7,142,906 (Yamashita et al., Nov. 28, 2006, “Optical Measurement Instrument for Living Body”) disclose an optical measurement instrument which applies visible-infrared light to several positions on a patient. U.S. Pat. No. 6,571,116 (Wake et al., May 27, 2003, “Medical Optical Imaging Scanner Using Multiple Wavelength Simultaneous Data Acquisition for Breast Imaging”) and U.S. Pat. No. 6,738,658 (Wake et al., May 18, 2004, “Medical Optical Imaging Scanner Using Multiple Wavelength Simultaneous Data Acquisition for Breast Imaging”) disclose a medical optical imaging device with an illumination source that directs emitted light into a breast positioned below a support surface.

U.S. patent application publication 20040092826 (Corbeil et al., May 13, 2004, “Method and Apparatus for Optical Imaging”) and U.S. Pat. No. 7,809,422 (Corbeil et al., Oct. 5, 2010, “Method and Apparatus for Optical Imaging”) disclose a platform for positioning a patient face down and a cavity into which one of the person's breasts is suspended for optical imaging. U.S. Pat. RE38800 (Barbour, Sep. 20, 2005, “NIR Clinical Opti-Scan System”) discloses three-dimensional optical imaging techniques for the detection and imaging of absorbing and/or scattering structures in body tissue. U.S. patent application publication 20070287897 (Faris, Dec. 13, 2007, “Optical Vascular Function Imaging System and Method for Detection and Diagnosis of Cancerous Tumors”) discloses an in-vivo optical imaging system and method of identifying unusual vasculature associated with tumors.

U.S. patent application publication 20090005692 (Intes et al., Jan. 1, 2009, “Optical Imaging Method for Tissue Characterization”) and U.S. Pat. No. 8,565,862 (Intes et al., Oct. 22, 2013, “Optical Imaging Method for Tissue Characterization”) disclose a method for detecting and characterizing abnormalities within biological tissue by characterizing optical properties of the tissue. U.S. Pat. No. 8,027,711 (Jones et al., Sep. 27, 2011, “Laser Imaging Apparatus with Variable Patient Positioning”) discloses a tabletop to support a patient in front-down position and an opening to permit a breast of the patient to be vertically pendant below the tabletop. U.S. Pat. No. 8,224,426 (Lilge et al., Jul. 17, 2012, “Optical Transillumination and Reflectance Spectroscopy to Quantify Disease Risk”) discloses spectroscopic tissue volume measurements with non-ionizing radiation to detect pre-disease transformations in tissue. U.S. patent application publication 20140236003 (Hielscher et al., Aug. 21, 2014, “Interfacing Systems, Devices, and Methods for Optical Imaging”) discloses an imaging interface with a plurality of concentric rings for diffuse optical tomography of breast tissue.

U.S. patent application publication 20140236021 (Islam, Aug. 21, 2014, “Near-Infrared Super-Continuum Lasers for Early Detection of Breast and Other Cancers”) and U.S. Pat. No. 9,993,159 (Islam, Jun. 12, 2018, “Near-Infrared Super-Continuum Lasers for Early Detection of Breast and Other Cancers”) disclose a system and method for using near-infrared or short-wave infrared light sources for early detection and monitoring of breast cancer. U.S. patent application publication 20180289264 (Islam, Oct. 11, 2018, “High Signal-to-Noise Ratio Light Spectroscopy of Tissue”) discloses a diagnostic system which delivers an optical beam to a nonlinear element that broadens a spectrum of the first optical beam to at least 10 nanometers through a nonlinear effect in the nonlinear element.

U.S. patent application publication 20150119665 (Barbour et al., Apr. 30, 2015, “Self-Referencing Optical Measurement for Breast Cancer Detection”) and U.S. Pat. No. 9,724,489 (Barbour et al., Aug. 8, 2017, “Self-Referencing Optical Measurement for Breast Cancer Detection”) discloses obtaining optical data from a pair of breasts, employing a simultaneous bilateral referencing protocol, and employing a self-referencing data analysis method. U.S. patent application publication 20160066811 (Mohamadi, Mar. 10, 2016, “Handheld and Portable Scanners for Millimeter Wave Mammography and Instant Mammography Imaging”) discloses an array of ultra-wide band radio frequency sensors for breast imaging.

U.S. patent application publication 20170007187 (Breneisen et al., Jan. 12, 2017, “Cancer Detector Using Deep Optical Scanning”) discloses Deep Optical Scanning (DEOS) for the detection of breast cancer and the determination of response to therapy. U.S. Pat. No. 9,597,046 (Goossen et al., Mar. 21, 2017, “Method and Device for Imaging Soft Body Tissue Using X-Ray Projection and Optical Tomography”) discloses breast imaging using X-ray projection techniques and optical tomography techniques. U.S. Pat. No. 9,770,220 (Stearns et al., Sep. 26, 2017, “Integrated Microtomography and Optical Imaging Systems”) discloses a rotating table that supports an imaging object, an optical stage, and separate optical and microtomography imaging systems.

U.S. patent application publication 20180070891 (Jepsen, Mar. 15, 2018, “Imaging With Infrared Imaging Signals”) discloses using an infrared imaging signal to image tissue. U.S. patent application publication 20180335753 (Jepsen et al., Nov. 22, 2018, “Co-Located Imaging and Display Pixel”) discloses an optical transformation engine coupled between an image pixel and a display pixel. U.S. patent application publication 20190072897 (Jepsen et al., Mar. 7, 2019, “Applications of Diffuse Medium Imaging”) discloses methods and an apparatus for imaging translucent materials. U.S. patent Ser. No. 10/215,636 (Fujii et al., Feb. 26, 2019, “Imaging Device Provided With Light Source That Emits Pulsed Light and Image Sensor”) discloses an imaging device with a light source that emits pulsed light at different wavelengths.

Fantini (2005), “Optical Spectroscopy and Imaging of Tissues”, NSF Award, 2005 (abstract only viewed), researched improved techniques for optical spectroscopy and imaging of biological tissues. Durduran et al. (2010), “Diffuse Optics for Tissue Monitoring and Tomography”, Reports on Progress in Physics, 2010, 73(7), 076701, discloses using near-infrared or diffuse optical spectroscopy to measure tissue hemodynamics. Soliman et al. (2010), “Functional Imaging Using Diffuse Optical Spectroscopy of Neoadjuvant Chemotherapy Response in Women with Locally Advanced Breast Cancer”, Clinical Cancer Research, Apr. 20, 2010, 15, 2605-2614, discloses functional imaging with tomographic near-infrared diffuse optical spectroscopy to measure tissue concentration of deoxyhemoglobin, oxyhemoglobin, percent water, and scattering power.

Khan (2013), “Image Reconstruction in Diffuse Optical Tomography With Sparsity Constraints”, NSF Award, 2013 (abstract only viewed), researched the use of sparsity-constrained regularization for solving the diffuse optical tomography inverse problem. Lutzweiler et al. (2013), “Optoacoustic Imaging and Tomography: Reconstruction Approaches and Outstanding Challenges in Image Performance and Quantification”, Sensors 2013, 13(3), 7345-7384, reviews optoacoustic imaging from image reconstruction and quantification perspectives. Tromberg et al. (2016), “Predicting Responses to Neoadjuvant Chemotherapy in Breast Cancer: ACRIN 6691 Trial of Diffuse Optical Spectroscopic Imaging”, Cancer Research, Aug. 15, 2016, 76(20), 5933-5944, investigates whether changes from baseline to mid-therapy in a diffuse optical spectroscopic imaging (DOSI)-derived imaging endpoint, the tissue optical index, predict pathologic complete response in women undergoing breast cancer neoadjuvant chemotherapy.

Grosenick et al. (2016), “Review of Optical Breast Imaging and Spectroscopy”, Journal of Biomedical Optics, September, 2016, 21(9), 091311 reviews the monitoring of neoadjuvant chemotherapy and breast cancer risk assessment via optical breast imaging and spectroscopy. Kim et al. (2016), “US-Localized Diffuse Optical Tomography in Breast Cancer: Comparison With Pharmacokinetic Parameters of DCE-MRI and With Pathologic Biomarkers”, BMC Cancer, Feb. 1, 2016, 16:50, discloses correlating parameters of ultrasonography-guided diffuse optical tomography with the pharmacokinetic features of dynamic contrast-enhanced MRI and pathologic markers of breast cancer. Ray et al. (2017), “A Systematic Review of Wearable Systems for Cancer Detection: Current State and Challenges”, Journal of Medical Systems, Oct. 2, 2017, 41(11), 180, reviews cancer detection using wearable systems, including sensor-based smart systems comprised of microcontroller, Bluetooth module, and smart phone.

Shokoufi et al. (2017), “Novel Handheld Diffuse Optical Spectroscopy Probe for Breast Cancer Assessment: Clinical Study”, Journal of Biomedical Science, 6(5), 34, discloses a hand-held continuous-wave radio-frequency modulated diffuse optical spectroscopy probe. Mabou et al. (2018), “Breast Cancer Detection Using Infrared Thermal Imaging and a Deep Learning Model”, Sensors, 2018, 18(9), 2799, discloses the use of infrared digital imaging for breast cancer detection, assuming thermal comparison between a healthy breast and a breast with cancer shows an increase in thermal activity in the precancerous tissues and areas surrounding developing breast cancer. Gunther et al. (2018), “Dynamic Diffuse Optical Tomography for Monitoring Neoadjuvant Chemotherapy in Patients with Breast Cancer”, Radiology, 287(3), June, 2018, 778-786, identifies dynamic optical imaging features associated with pathologic response in patients with breast cancer during neoadjuvant chemotherapy.

Qiu (2018), “Implantable Ultra-low Power VO2 MEMS Scanner Based Surface-Enhanced Raman Spectroscope for Wide-field Tumor Imaging in Free Moving Small Animals”, NSF Award, 2018 (abstract only viewed) discloses tumor-targeting surface enhanced Raman scattering nanoparticles based multiplexed Raman spectroscopy. Moreno et al. (2019), “Evaluation on Phantoms of the Feasibility of a Smart Bra to Detect Breast Cancer in Young Adults”, Sensors, Dec. 12, 2019, 19(24), 5491, discloses the use of breast tissue phantoms to investigate the feasibility of quantifying breast density and detecting breast cancer tumors using a smart bra. Koomson (2019), “PFI-TT: A Noninvasive Biological Research Tool for Measurement of Tissue and Cerebral Oxygenation,” NSF Award, 2019 (abstract only viewed) researches compact wearable devices with advanced NIRS capability.

### SUMMARY OF THE INVENTION

The invention disclosed herein is a smart bra which can serve as a wearable device for optical breast imaging and spectroscopic tissue analysis. It has several potential advantages over the current art. As an advantage over x-ray mammography, a wearable optical breast imaging device does not expose a women to potentially harmful ionizing radiation and does not require the discomfort of breast compression. As an advantage over current stationary optical imaging devices, an optical breast imaging bra can enable a close fit (even for breasts of different sizes and shapes) to ensure good optical communication between the breast and the light emitters and light receivers of the device. This can also enable tracking absolute tissue locations to evaluate changes in breast tissue at selected locations over time.

This invention can be embodied in a wearable optical breast imaging device (e.g. “smart bra”) with: (a) at least one cup which is configured to be worn on at least one breast; (b) multiple light emitters on the interior of the cup which emit light beams into the breast tissue at different times; and (c) multiple light receivers on the interior of the cup which receive the light beams after they have been transmitted through breast tissue. Changes in the spectra of light energy caused by transmission of the light energy through the breast tissue are analyzed in order to diagnose whether there is abnormal tissue within the breast and, if so, to identify the location of abnormal tissue within the breast.

In an example, spectral changes in light transmitted through breast tissue along at least three intersecting three-dimensional vectors are analyzed to identify the three-dimensional location of abnormal tissue within the breast. Although light energy is diffused through the depth of breast tissue, joint three-dimensional analysis of light transmitted through multiple intersecting vectors between multiple pairs of light emitters and light receivers can increase the locational precision of spectroscopic analysis in order to identify and locate abnormal tissue. In an example, this device can be embodied in a bra with two such cups which can be used for breast cancer screening without exposure to potentially-harmful radiation.

A wearable optical breast imaging device will probably not be as accurate for detecting breast tissue anomalies as x-ray mammography or MR imaging in the next couple years. However, there is rapid progress underway in the miniaturization of spectroscopic sensors and their increased incorporation into wearable devices. With this continued progress in the years to come, a wearable optical breast imaging device can evolve into a very useful, safe, painless, and low-cost modality for frequent regular breast imaging and preliminary screening.

### INTRODUCTION TO THE FIGURES

FIGS. 1 through 9 show a wearable optical breast imaging device with at least one cup, an array of light emitters, and an array of light receivers.

FIGS. 1 through 3 show this device at different times as different light emitters in a ring are activated.

FIGS. 4 through 6 show this device at different times as different light receivers in a ring are activated.

FIGS. 7 through 9 show how analysis of spectral changes of light transmitted between different light emitter and receiver pairs can be used to locate an abnormal mass within breast tissue.

### DETAILED DESCRIPTION OF THE FIGURES

FIGS. 1 through 9 show multiple front views of an example of a wearable optical breast imaging device comprising: at least one cup 101 which is configured to be worn on at least one breast 701; an array of light emitters (including light emitters 102, 103, and 104) on the interior of the cup which emit light beams into the breast at different times; and an array of light receivers (including light receivers 105, 106, and 107) on the interior of the cup which receive the light beams after they have been transmitted through the breast tissue, wherein changes in the spectra of the light beams caused by transmission of the light beams through the breast tissue are spectroscopically analyzed in order to diagnose whether there is abnormal tissue 702 within the breast and, if so, to identify the location of the abnormal tissue within the breast.

FIGS. 1 through 6 show this device at six different times wherein different pairs of light emitters and light receivers are activated to record spectral changes in light transmitted through the breast tissue along different three-dimensional vectors. Joint analysis of the spectral changes of light beams traveling through the breast tissue along different three-dimensional vectors can identify whether there is abnormal tissue within the breast and, if so, where the abnormal tissue is located. Although light energy is significantly diffused through the depth of breast tissue, joint three-dimensional analysis of light transmitted through multiple intersecting vectors between multiple pairs of light emitters and light receivers can increase the accuracy and locational precision of spectroscopic analysis in order to identify and locate abnormal tissue.

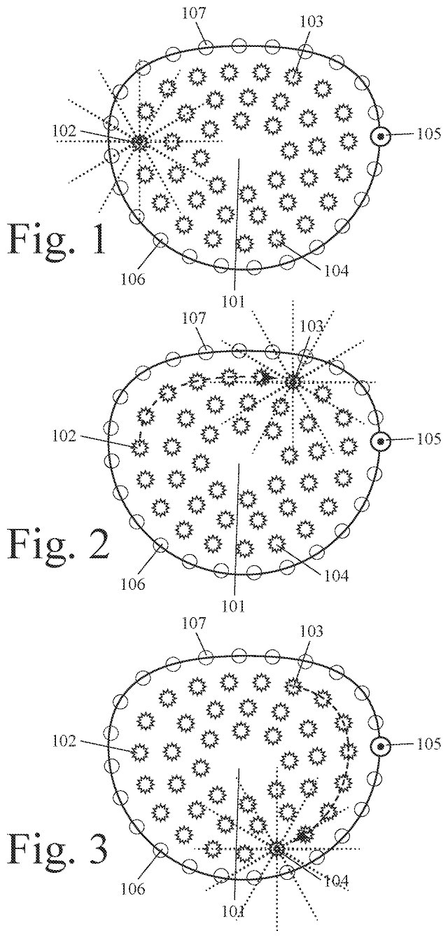

FIGS. 1 through 3 show an example of this device with activation of different light emitters at different times, wherein light from the different emitters is received by the same light receiver. In this series, different light emitters comprise a ring of light emitters which are activated in a clockwise manner around the ring. FIG. 1 shows this device at a first point in time when light emitter 102 is activated to emit light and light receiver 105 is activated to receive this light. FIG. 2 shows this device at a second point in time when light emitter 103 is activated to emit light and light receiver 105 is activated to receive this light. FIG. 3 shows this device at a second point in time when light emitter 104 is activated to emit light and light receiver 105 is activated to receive this light.

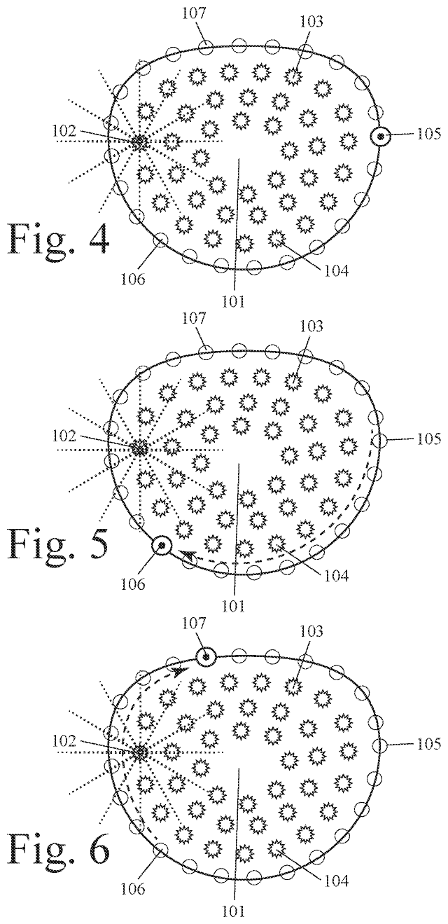

FIGS. 4 through 6 show an example of this device with activation of a light emitter, wherein light from the light emitter is received by different light receivers at different times. In this series, different light receivers comprise a ring of light receivers which are activated in a clockwise manner around the ring. FIG. 4 shows this device at a first point in time when light emitter 102 is activated to emit light and light receiver 105 is activated to receive this light. FIG. 5 shows this device at a second point in time when light emitter 102 is activated to emit light and light receiver 106 is activated to receive this light. FIG. 6 shows this device at a second point in time when light emitter 102 is activated to emit light and light receiver 107 is activated to receive this light. One or both of these patterns (or other patterns) of sequential activation of different pairs of light emitters and receivers can be used for three-dimensional analysis of changes in light spectra through different locations within breast tissue.

In the example shown in FIGS. 1 through 6, light emitters are activated at different times around a ring in a clockwise sequence. In another example, light emitters can be activated at different times around a ring in a counter-clockwise sequence. In another example, light emitters can be activated at different times along a radial spoke in a spoke-and-ring array. In another example, light emitters can be activated at different times along a radial spoke in an inward-moving sequence. In another example, light emitters can be activated at different times along a radial spoke in an outward-moving sequence. In another example, light emitters can be activated sequentially in an undulating and/or sinusoidal pattern. In another example, light emitters can be activated sequentially in a spiral and/or helical pattern.

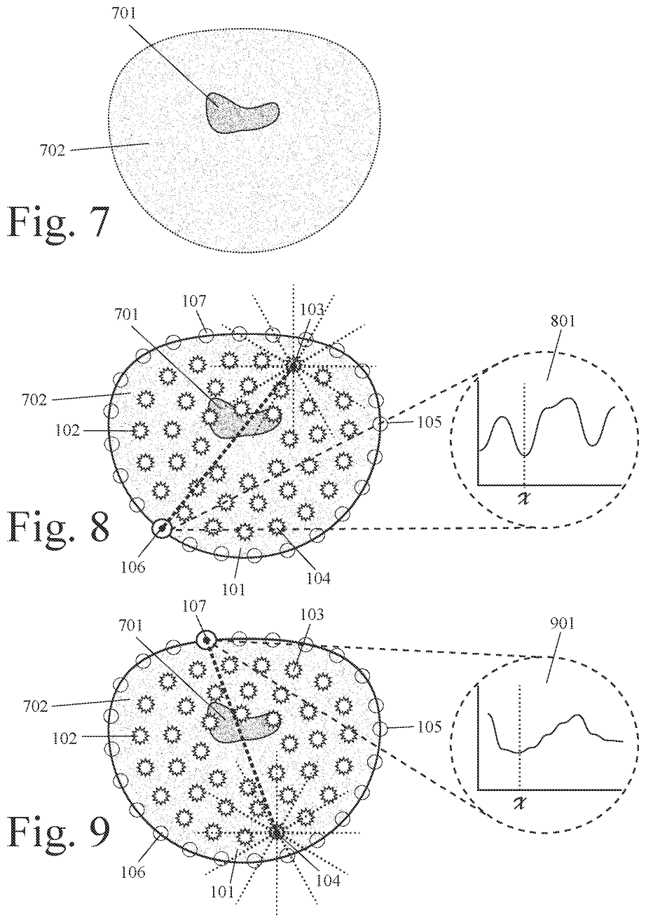

FIGS. 7 through 9 show an example of how analysis of spectral changes of light transmitted along intersecting three-dimensional vectors between different light emitter and receiver pairs can be used to identify the location of an abnormal mass within breast tissue. FIG. 7 shows an abnormal mass 701 within breast tissue 702. FIG. 8 shows a device at a first period of time comprising a cup 101 covering the breast. This device further comprises an array of light emitters (including 102, 103, and 105) on the interior of the cup and an array of light receivers (including 104, 105, and 106) on the interior of the cup. In FIG. 8, light emitter 103 has been activated to emit light into the breast and light receiver 106 has been activated to receive this light after it has been transmitted through breast tissue. Graph 801 shows the spectral distribution of light from light emitter 103 received by light receiver 106, including a trough at wavelength x which is associated with abnormal tissue. In an example, a trough can be associated with selective absorption of light at this wavelength by a high level of collagen, hemoglobin, deoxyhemoglobin, oxyhemoglobin, lipids, and oxygen which is associated with abnormal tissue.

FIG. 9 shows this device at a second period of time. In FIG. 9, light emitter 104 has been activated to emit light into the breast tissue and light receiver 107 has been activated to receive this light after it has been transmitted through the breast tissue. Graph 901 shows the spectral distribution of light from light emitter 104 received by light receiver 106, including a trough at wavelength x which is associated with abnormal tissue. Multi-dimensional analysis of where the light vectors between two (or more) pairs of light emitters and receivers intersect can identify the location of an abnormal mass 701. In an example, wearable optical breast imaging device can comprise a bra, with two cups such as the one shown here, wherein this bra can be used for breast cancer screening without exposure to harmful radiation.

FIGS. 1 through 9 can also be said to show a wearable optical breast imaging device comprising: at least one cup which is configured to be worn on at least one breast; an array of light emitters on the interior of the cup which emit light beams into the breast tissue at different times; and an array of light receivers on the interior of the cup which receive the light beams after they have been transmitted through the breast tissue, wherein changes in the spectra of the light beams caused by transmission of the light beams through the breast tissue are analyzed in order to diagnose whether there is abnormal tissue within the breast and, if so, to identify the location of abnormal tissue within the breast.

FIGS. 1 through 9 can also be said to show a wearable optical breast imaging device comprising: at least one cup which is configured to be worn on at least one breast; wherein the cup further comprises a first light transmission pair comprising a first light emitter and a first light receiver and wherein the first light transmission pair transmits light through the breast tissue along a first vector at a first time; wherein the cup further comprises a second light transmission pair comprising a second light emitter and a second light receiver and wherein the second light transmission pair transmits light through the breast tissue along a second vector at a second time; wherein the cup further comprises a third light transmission pair comprising a third light emitter and a third light receiver and wherein the third light transmission pair transmits light through the breast tissue along a third vector at a third time; wherein the three vectors intersect in three-dimensional space within the breast tissue; and wherein changes in the spectra of the light beams from the three light transmission pairs caused by transmission of the light beams through the breast tissue are jointly analyzed in order to determine whether there is abnormal tissue at the location where the three vectors intersect.

In an example, the wearable optical breast imaging device shown in FIGS. 1 through 9 can further comprise an energy source, data processor, and data transmitter. In an example, an energy source, data processor, and data transmitter can be located in a back portion of a bra, near a strap connection at the back, and in electromagnetic communication with one or more cups through wires. In an example, wearable optical breast imaging device can be in wireless communication with a smart phone and/or smart watch in order to control its activation and display results. Relevant variations discussed elsewhere in this disclosure or in priority linked disclosures can also be applied to the example shown in FIGS. 1 through 9.

In the example shown in FIGS. 1 through 9, a wearable optical breast imaging device has multiple (e.g. three) concentric rings of light emitters on the interior surface of a cup and an arcuate array (e.g. ring) of light receivers around the interior perimeter of the cup. In an example, a wearable optical breast imaging device can have multiple (nested) rings of light emitters and light receivers on the interior surface of the cup. In an example, a wearable optical breast imaging device can have multiple (nested) rings with alternating light emitters and light receivers on the interior surface of the cup. In this example, a wearable optical breast imaging device can have a spoke-and-ring array with (alternating) light emitters and light receivers on the interior surface of the cup and an arcuate array (e.g. ring) of light receivers around the interior perimeter of the cup. In this example, a wearable optical breast imaging device can have a latitude-and-longitude array with (alternating) light emitters and light receivers on the interior surface of the cup and an arcuate array (e.g. ring) of light receivers around the interior perimeter of the cup. In this example, a wearable optical breast imaging device can have a grid or matrix of (alternating) light emitters and light receivers on the interior surface of the cup and an arcuate array (e.g. ring) of light receivers around the interior perimeter of the cup.

In an example, a wearable optical breast imaging device can have an array of different light emitters which emit light with different colors and/or wavelengths at the same time. In an example, a wearable optical breast imaging device can have an array of light emitters which emit light with different colors and/or wavelengths at different times. In an example, a wearable optical breast imaging device can have an array of different light emitters which emit light at different angles and/or from different locations at the same time. In an example, a wearable optical breast imaging device can have an array of different light emitters which emit light at different angles and/or from different locations at different times.

In an example, light emitters of this device can be selected from the group consisting of: light emitting diodes (LEDs), coherent light sources, organic light emitting diodes (OLEDs), lasers, laser diodes, infrared light-energy emitters, multi-wavelength sources, resonant cavity light emitting diodes (RCLEDs), super-luminescent light emitting diodes (SLEDs), and ultraviolet light-energy emitters. In an example, this device can comprise an array of fiber optic pathways which transmit light from light emitters to different locations on a breast cup (at different times), from which the light enters the breast at different locations. In an example, this device can comprise an array of flexible silicone-based (e.g. polydimethylsiloxane) fiber optic pathways which transmit light to different locations on a breast cup (at different times). In an example, this device can further comprise one or more moving micromirrors, prisms, or lenses which direct light (e.g. along different fiber optic pathways) to different locations on a breast cup at different times.

In an example, an array of light emitters can include a plurality of light emitters which are distributed along the lower perimeter of a breast cup. In an example, an array of light emitters can include a plurality of light emitters distributed along the upper perimeter of a breast cup. In an example, an array of light emitters can have semi-circular or semi-elliptical shape. In an example, an array of light emitters can have a conic section shape. In an example, an array of light emitters can comprise rings of light emitters. In an example, an array of light emitters can comprise concentric rings of light emitters. In an example, an array of light emitters can comprise nested rings of light emitters. In an example, an array of light emitters can comprise a ring and spoke array of light emitters. In an example, an array of light emitters can comprise a latitude and longitude array of light emitters. In an example, an array of light emitters can comprise a radial grid, mesh, or matrix of light emitters. In an example, an array of light emitters can comprise a polar coordinate (e.g. spoke and ring) array of light emitters.

In an example, an array of light receivers can include a plurality of light receivers which are distributed along the lower perimeter of a breast cup. In an example, an array of light receivers can include a plurality of light receivers distributed along the upper perimeter of a breast cup. In an example, an array of light receivers can have semi-circular or semi-elliptical shape. In an example, an array of light receivers can have a conic section shape. In an example, an array of light receivers can comprise rings of light receivers. In an example, an array of light receivers can comprise concentric rings of light receivers. In an example, an array of light receivers can comprise nested rings of light receivers. In an example, an array of light receivers can comprise a ring and spoke array of light receivers. In an example, an array of light receivers can comprise a latitude and longitude array of light receivers. In an example, an array of light receivers can comprise a radial grid, mesh, or matrix of light receivers. In an example, an array of light receivers can comprise a polar coordinate (e.g. spoke and ring) array of light receivers.

In an example, an array of light emitters can include one or more near-infrared light emitters. In an example, an array of light emitters can include one or more infrared light emitters. In an example, an array of light emitters can include one or more red light emitters. In an example, an array of light emitters can include one or more green light emitters. In an example, different light emitters in an array can emit light at different wavelengths at the same time. In an example, different light emitters in an array can emit light at different wavelengths at different times. In an example, the same light emitter in an array can emit light at different wavelengths at different times. In an example, an array of light emitters can include both near-infrared light emitters and green light emitters.

In an example, different light emitters in an array of light emitters can emit light into breast tissue from different locations at different times. In an example, different light emitters in an array of light emitters can emit light with different intensities, colors/wavelengths, or durations. In an example, different light emitters in an array of light emitters can emit light with different intensities, colors/wavelengths, or durations at different times. In an example, the same light emitter in an array of light emitters can emit light with different intensity, color/wavelength, or duration at different times. In an example, the intensity, color/wavelength, or duration of light energy emitted from a light emitter can be modulated over time. In an example, different light emitters in an array of light receivers can receive light transmitted through breast tissue from different locations at different times.

In an example, changes in the spectrum of light energy transmitted through breast tissue can be analyzed in order to identify abnormal vasculature, lymphamatics, abnormal extracellular matrix, and/or vascular sprouting. In an example, changes in the spectrum of light energy transmitted through breast tissue can be analyzed in order to identify abnormal levels of: collagen; hemoglobin, deoxyhemoglobin, and/or oxyhemoglobin; lipids; and/or oxygen saturation. In an example, changes in the spectra of beams of light energy transmitted through breast tissue between multiple pairs of light emitters and light receivers can be jointly analyzed (and triangulated) in order to identify the specific locations abnormal vasculature, lymphamatics, abnormal extracellular matrix, and/or vascular sprouting in breast tissue. In an example, changes in the spectra of beams of light energy transmitted through breast tissue between multiple pairs of light emitters and light receivers can be jointly analyzed (and triangulated) in order to identify the specific locations of abnormal levels of: collagen; hemoglobin, deoxyhemoglobin, and/or oxyhemoglobin; lipids; and/or oxygen saturation. In an example, spectroscopic analysis of light transmitted through breast tissue can comprise diffuse optical imaging, broadband spectroscopy, and/or diffuse correlation spectroscopy.

In an example, different pairs of light emitters and light receivers, at different locations on a breast cup, can be activated to transmit light into breast tissue and receive the light transmitted through the breast tissue, respectively, at different times. In an example, changes in the spectra of light transmitted through breast tissue between different pairs of light emitters and light receivers can be jointly analyzed (e.g. triangulated) to identify the location of abnormal tissue within the three-dimensional space of the breast. In an example, spectral troughs or peaks at one or more selected frequencies can be identified in intersectional locations between two or three pairs of light emitters and light receivers in order to identify the location of abnormal tissue in two-dimensional or three-dimensional space. In an example, spectral troughs or peaks associated with abnormal levels of collagen, hemoglobin, deoxyhemoglobin, oxyhemoglobin, lipids, or oxygen can be identified in intersectional locations between two or three pairs of light emitters and light receivers in order to identify the location of abnormal tissue in two-dimensional or three-dimensional space.

While diffusion of light between one light emitter and light receiver pair may cause results to be imprecise, combined analysis of spectral changes between multiple light emitter and light receiver pairs with intersecting light pathways may enable more precise identification and locational identification of abnormal tissue location. In an example, a spoke and ring array of light emitters and light receivers may enable creation of a three-dimensional spectroscopic map of breast tissue in order to diagnose abnormal masses.

In an example, a wearable optical breast imaging device can comprise: a cup which is worn on a person's breast; a concentric and/or nested ring array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a concentric and/or nested ring array of light energy emitters on the interior of the cup and a light energy receiver on the interior of the cup, wherein different light energy emitters in the array emit light energy into the breast at varying and/or modulated wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a conic-section-shaped array of light energy emitters around a first portion of the perimeter of the cup and a conic-section-shaped array of light energy receivers around a second portion of the perimeter of the cup, wherein the first portion is diametrically-opposite the second portion, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a conic-section-shaped array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a conic-section-shaped array of light energy emitters which are distributed along a first side of the perimeter of the cup and an array of light energy receivers which are distributed along a second side of the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a conic-section-shaped array of light energy emitters which are distributed along a first portion the perimeter of the cup, wherein the light energy emitters emit light energy into the breast, and wherein the first portion spans from the 10 o'clock to 2 o'clock positions (along the top of the cup); and an array of light energy receivers which are distributed along a second portion of the cup, wherein the light energy receivers receive the light energy after it has been transmitted through breast tissue, wherein the first portion spans from the 4 o'clock to 8 o'clock positions (along the bottom of the cup), and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of LEDs on the interior of the cup which emit light energy into the breast from different locations at different times; and a light energy detector on the interior of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; a convex array of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of light energy emitters on the perimeter of the cup and a convex array of light energy receivers on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a convex array of light energy emitters on the upper perimeter of the cup and a light energy receiver on the lower perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; a convex array of optical transmission pathways which spans at least three-quarters of the interior of the cup and which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a convex grid, matrix, or array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; a convex radial grid, mesh, or matrix of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a first light energy emitter on the (base) perimeter of the cup which emits light into the breast with a wavelength in the range of 650 to 700 nm; a second light energy emitter on the (base) perimeter of the cup which emits light into the breast with a wavelength in the range of 700 nm to 750 nm; a third light energy emitter on the (base) perimeter of the cup which emits light into the breast with a wavelength in the range of 750 nm to 800 nm; and one or more light energy receivers on the (base) perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the cup which emits light into the breast with a first wavelength at a first time; a second light energy emitter on the cup which emits light into the breast with a second wavelength at a second time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a first light energy emitter on the interior of the cup which emits intensity-modulated light into the breast with a wavelength in the range of 650 to 750 nm; a second light energy emitter on the interior of the cup which emits intensity-modulated light into the breast with a wavelength in the range of 750 nm to 850 nm; a third light energy emitter on the interior of the cup which emits intensity-modulated light into the breast with a wavelength in the range of 850 nm to 950 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the interior of the cup which emits light into the breast with a wavelength in the range of 600 to 800 nm; a second light energy emitter on the interior of the cup which emits light into the breast with a wavelength in the range of 800 nm to 1000 nm; a third light energy emitter on the interior of the cup which emits light into the breast with a wavelength in the range of 1000 nm to 1200 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the interior of the cup which emits light into the breast with a wavelength in the range of 600 to 900 nm at a first time; a second light energy emitter on the interior of the cup which emits light into the breast with a wavelength in the range of 900 nm to 1200 nm at a second time; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the interior of the cup which emits light into the breast with a wavelength in the range of 600 to 900 nm at a first time; a second light energy emitter on the interior of the cup which emits light into the breast with a wavelength in the range of 900 nm to 1200 nm at a second time; a third light energy emitter on the interior of the cup which emits light into the breast with a wavelength in the range of 1200 nm to 1500 nm at a third time; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a first light energy emitter on the interior of the cup which emits a continuous beam of light into the breast from a first location at a first wavelength at a first time; a second light energy emitter on the interior of the cup which emits short pulses of light into the breast from a second location at a second wavelength at a second time; and a light energy receiver on the interior of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the perimeter of the cup which emits light into the breast with a wavelength in the range of 650 to 750 nm; a second light energy emitter on the perimeter of the cup which emits light into the breast with a wavelength in the range of 750 nm to 850 nm; a third light energy emitter on the perimeter of the cup which emits light into the breast with a wavelength in the range of 850 nm to 950 nm; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a first light energy emitter on the perimeter of the cup which emits light into the breast with a wavelength in the range of 600 to 700 nm at a first time; a second light energy emitter on the perimeter of the cup which emits light into the breast with a wavelength in the range of 700 nm to 800 nm at a second time; a third light energy emitter on the perimeter of the cup which emits light into the breast with a wavelength in the range of 800 nm to 900 nm at a third time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a first light energy emitter on the perimeter of the cup which emits light into the breast with a first modulated wavelength at a first time; a second light energy emitter on the perimeter of the cup which emits light into the breast with a second modulated wavelength at a second time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a grid, mesh, or matrix of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a hub-and-spoke array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a latitude-and-longitude array of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a multi-layer cup which is worn on a person's breast; a light energy emitter; a moving mirror, prism, or lens which is in optical communication with the light energy emitter; a plurality of flexible fiber optic pathways between interior and exterior layers of the multi-layer cup; wherein different flexible fiber optic pathways are in optical communication with the moving mirror, prism, or lens at different times as the mirror, prism, or lens moves; and wherein different flexible fiber optic pathways transmit light energy from the light energy emitter into the breast from different locations on the interior of the multi-layer cup at different times; and one or more light energy receivers on the multi-layer cup which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can be embodied in: a cup with an inner layer and an outer layer which is worn on a person's breast; a light energy emitter; a plurality of polydimethylsiloxane (PDMS) optical pathways between the inner layer and the outer layer which guide light from the light energy emitter to different locations on the cup, from which locations the light energy is transmitted into the person's breast; and a light energy receiver which receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a light energy emitter; an array of PDMS-based optical pathways in the cup which channel light from the light energy emitter to different locations on the interior of the cup from which the light is transmitted into the breast at different times; and a light energy receiver on the interior of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a light energy receiver on the lower perimeter of the cup, and a nested-ring array of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a light energy receiver on the lower perimeter of the cup, and an array of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; a moving micromirror, prism, or lens; a nested-ring array of optical pathways spanning a portion of the cup which are in optical communication with the moving micromirror, prism, or lens; a light energy emitter, wherein light energy from the light energy emitter is guided to be transmitted into the breast from different positions on the interior of the cup at different times by the moving micromirror, prism, or lens and the plurality of flexible optical pathways; and a light energy receiver; wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a moving micromirror, prism, or lens; an array of optical pathways spanning a portion of the cup which are in optical communication with the moving micromirror, prism, or lens; a light energy emitter, wherein light energy from the light energy emitter is guided to be transmitted into the breast from different positions on the interior of the cup at different times by the moving micromirror, prism, or lens and the plurality of flexible optical pathways; and a light energy receiver; wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a plurality of light energy emitters on a lower portion (e.g. the lower half) of the cup, wherein the plurality of light energy emitters emit light energy into the breast sequentially (at different locations at different times); and one or more light energy receivers on an upper portion (e.g. the upper half) of the cup, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a plurality of light energy emitters on the cup which emit light energy into the breast within a range of 600 nm to 1,000 nm; and one or more light energy emitters on the cup which receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a plurality of light energy emitters on the cup which emit light energy into the breast, wherein different light energy emitters in the array emit light energy into the breast at different intensities; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup with an inner layer and an outer layer which is worn on a person's breast; a plurality of light energy emitters; a plurality of flexible optical pathways between the inner layer and the outer layer which guide light from the light energy emitters to different locations on the cup, from which locations the light energy is transmitted into the person's breast; and a plurality of light energy receivers which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is configured to be worn on a person's breast, wherein the cup further comprises a convex elastic fabric portion which covers at least part of the front of the breast; a plurality of pairs of light energy emitters and light energy receivers on the convex elastic fabric portion, wherein light energy emitters are activated sequentially to emit light energy into the breast at different times, wherein light energy receivers receive the light energy transmitted through breast tissue, wherein a light energy emitter and a light energy receiver in a transmitting and receiving pair are located on opposite sides of the breast, and wherein light energy transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a polar-coordinate array of light energy emitters on the interior of the cup which emit light energy into the breast; a light energy emitter on the interior of the cup which receives light energy from the light emitters after it has been transmitted through breast tissue; wherein the light energy is analyzed to create an image of the breast, identify the molecular and/or cellular composition of the breast, and/or identify the locations and/or shapes of selected types of tissue within the breast; and a data processor and data transmitter. In another example, a wearable optical breast imaging device can be embodied in: a cup which is worn on a person's breast; a radial-spoke ring array of light energy emitters on the interior of the cup and one or more light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a red-light laser on the interior of the cup which emits light energy into the breast; an infrared laser on the interior of the cup which emits light energy into the breast; and one or more light energy receivers on the interior of the cup which receive light energy from the red-light and infrared lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can comprise: a cup which is worn on a person's breast; a ring of light energy emitters and light energy receivers around at least half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a ring of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; a ring of light energy emitters on the interior of the cup; and a ring of light energy receivers on the interior of the cup, wherein the light energy emitters emit light energy into the breast, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; a semi-circular, semi-elliptical, or other conic section array of light energy emitters and light energy receivers on the lower perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a sinusoidal array of light energy emitters on the interior of the cup which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an alternating sequence of light energy emitters and light energy receivers around the (base) perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an arcuate array of light energy emitters and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an arcuate array of light energy emitters on the interior of the cup and a light energy receiver on the interior of the cup, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an arcuate grid, mesh, or matrix of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of at least fifty light energy emitters on the (base) perimeter of the cup which emit light energy into the breast from different locations at different times; and a light energy receiver on the (base) perimeter of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of at least fifty light energy emitters on the interior of the cup which emit light energy into the breast from different locations at different times; and an array of at least fifty light energy receivers on the (base) perimeter of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of at least ten light energy emitters on the interior of the cup which emit light energy into the breast from different locations at different times; and a light energy receiver on the (base) perimeter of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of at least ten light energy emitters on the upper half of the cup which emit light energy into the breast from different locations at different times; and a light energy receiver on the lower half of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of continuous wave light emitters on the interior of the cup which emit light energy into the breast; and an array of light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can be embodied in: an elastic and/or stretchable fabric cup which is worn on a person's breast; an array of elastic light energy pathways which are part of the cup and transmit light energy into the breast from different locations on the interior of the cup; and an array of light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of fiber optic light energy emitters on the interior of the cup and a light energy receiver on the interior of the cup, wherein light energy emitters in the array emit light energy into the breast at varying and/or modulated wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of fiber optic light-transmitting pathways on the cup, wherein different fiber optic light-transmitting pathways sequentially emit light energy into the breast at different locations; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein phase changes in the light energy transmitted through breast tissue are analyzed to identify levels of one or more substances selected from the group consisting of: collagen; hemoglobin, deoxyhemoglobin, and/or oxyhemoglobin; lipids; oxygen saturation; and water.

In an example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the absorption, scattering, and/or transmission of the light energy through the breast are analyzed to identify abnormal levels of one or more substances selected from the group consisting of: collagen; hemoglobin, deoxyhemoglobin, and/or oxyhemoglobin; lipids; oxygen saturation; and water. In an alternative example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the absorption, scattering, and/or transmission of the light energy through the breast are analyzed to create a map of the levels of one or more substances selected from the group consisting of: collagen; hemoglobin, deoxyhemoglobin, and/or oxyhemoglobin; lipids; oxygen saturation; and water.

For example, a wearable optical breast imaging device can comprise: a cup which is worn on a person's breast; an array of light energy emitters and light energy receivers which are distributed along the lower perimeter of the cup between the 4 o'clock and 8 o'clock locations on the perimeter, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array of light energy emitters and light energy receivers which are distributed along the lower perimeter of the cup spanning the 3 o'clock and 9 o'clock locations on the perimeter, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters and light energy receivers which are distributed across the upper and lower portions of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of light energy emitters and light energy receivers which are distributed in spokes around the convex interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of light energy emitters around the (base) perimeter of the cup, wherein the light energy emitters sequentially emit light energy into the breast; and an array of light energy receivers around the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an array of light energy emitters on a first quadrant of the perimeter of the cup, wherein the light energy emitters sequentially emit light energy into the breast; and an array of light energy receivers on a second quadrant of the perimeter of the cup, wherein the first quadrant is diametrically-opposite the second quadrant, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of light energy emitters on interior of the cup which emit laser pulses into the breast; one or more light energy receivers on the perimeter of the cup which receive light energy transmitted through breast tissue, wherein broadening of the laser pulses due to transmission through breast tissue is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can be embodied in: a cup which is worn on a person's breast; an array of light energy emitters on the cup which emits light energy into the breast; one or more light energy receivers on the perimeter of the cup which receive light energy transmitted through breast tissue, wherein light transmitted through breast tissue is analyzed using diffuse correlation spectroscopy. In an example, a wearable optical breast imaging device can comprise: an elastic and/or stretchable fabric cup with an opaque layer which is worn on a person's breast; an array of light energy emitters on the cup, interior relative to the opaque layer; and an array of light energy receivers on the interior of the cup, interior relative to the opaque layer, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup with an opaque layer which is worn on a person's breast; an array of light energy emitters on the interior of the cup; and an array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup with an opaque layer which is worn on a person's breast; an array of light energy emitters on the perimeter of the cup; and a light energy receiver on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of light energy emitters which are evenly distributed around at least one-half of the base perimeter of the cup, wherein the light energy emitters sequentially emit light energy into the breast from different locations at different times; and an array of light energy receivers which are evenly distributed around at least one-half of the base perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of light energy emitters which collectively span at the entire interior surface area of the cup and which emit light energy into the breast from different locations at different times; one or more light energy emitters on the interior of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In another example, a wearable optical breast imaging device can comprise: a cup which is worn on a person's breast; an array of matched pairs of light energy emitters and light energy receivers which is distributed around at least 50% of the (base) perimeter of the cup, wherein a light energy emitter in a matched pair emits light energy into the breast and a light energy receiver in the matched pair receives receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of matched pairs of light energy emitters and light energy receivers which is distributed around between 45% and 75% of the (base) perimeter of the cup, wherein a light energy emitter in a matched pair emits light energy into the breast and a light energy receiver in the matched pair receives receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; an array of matched pairs of light energy emitters and light energy receivers on the interior of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 1 cm and 3 cm apart, and wherein light energy transmitted through breast tissue is analyzed in order to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an array of matched pairs of light energy emitters and light energy receivers on the (base) perimeter of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 1 cm and 3 cm apart, and wherein light energy transmitted through breast tissue is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an array of matched pairs of light energy emitters and light energy receivers which is distributed around between 25% and 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is diametrically-opposite the light energy emitter in the matched pair; and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of matched pairs of light energy emitters and light energy receivers which is distributed around between 45% and 75% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is in same quadrant of the cup as the light energy emitter in the matched pair; and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of pairs of light energy emitters and light energy receivers around the (base) perimeter of the cup, wherein the a light energy receiver in a pair is diametrically-opposite a light energy emitter in the pair, wherein the light energy emitter in a pair emits light energy into the breast, wherein the light energy receiver in the pair receives the light energy after it has been transmitted through breast tissue, wherein different pairs are activated to emit and receive light at different times, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of pairs of light energy emitters and light energy receivers on the interior of the cup, wherein a light energy emitter in a pair emits light energy into the breast and a light energy receiver in the pair receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: an elastic and/or stretchable fabric cup which is worn on a person's breast; an array of PDMS-based light energy pathways which are part of the cup and transmit light energy into the breast from different locations on the interior of the cup; and a light energy receiver on the lower perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of polydimethylsiloxane (PDMS) light-transmitting pathways on the cup, wherein different light-transmitting pathways sequentially emit light energy into the breast at different locations; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: an elastic and/or stretchable fabric cup which is worn on a person's breast; an array of silicone-based light energy pathways which are part of the cup and transmit light energy into the breast from different locations on the interior of the cup; and a light energy receiver on the perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of time-domain light emitters on the (base) perimeter of the cup which emit short pulses of light energy into the breast; and an array of light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of triads, wherein each triad comprises two light energy emitters and one light energy receiver, and wherein the array of triads is distributed around the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of triads; wherein each triad comprises a first light energy emitter which emits light energy into the breast at a first time, a second light energy emitter which emits light energy into the breast at a second time, and a light energy receiver; wherein the array of triads is distributed around at least 50% of the (base) perimeter of the cup, wherein a light energy receiver receives light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an upper arcuate array of light energy emitters on the upper interior surface of the cup which emit light into an upper portion of the breast; a lower arcuate array of light energy emitters on the lower interior surface of the cup which emit light into a lower portion of the breast; an upper arcuate array of light energy receivers on the upper interior surface of the cup which receive light energy after it has been transmitted through breast tissue; and a lower arcuate array of light energy emitters on the lower interior surface of the cup which receive light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an upper arcuate array of light energy emitters on the upper perimeter of the cup which emit light into an upper portion of the breast; a lower arcuate array of light energy emitters on the lower perimeter of the cup which emit light into a lower portion of the breast; and an arcuate array of light energy receivers on the perimeter of the cup which receive light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an upper perimeter of the cup which comprises an arcuate hem, strap, fold, wire, or cuff; a lower perimeter of the cup which comprises an arcuate hem, strap, fold, wire, or cuff; one or more light energy emitters on the lower perimeter which emit light energy into the breast; and one or more light energy receivers on the lower perimeter which receive light energy transmitted through breast tissue, wherein broadening of the laser pulses due to transmission through the breast is analyzed to create an image of the breast, identify the molecular and/or cellular composition of the breast, and/or identify the locations and/or shapes of selected types of tissue within the breast; and a data processor and data transmitter.

For example, a wearable optical breast imaging device can be embodied in: a cup which is worn on a person's breast; at least one light energy emitter on the interior of the cup which emits light into the breast; and at least one light energy receiver on the interior of the cup which receives the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; concentric rings of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; one or more continuous wave light emitters on the interior of the cup which emit light energy into the breast; and one or more light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; one or more flexible fiber optic light energy emitters which are integrated into the cup; and one or more light energy receivers on the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an example, a wearable optical breast imaging device can comprise: a cup which is worn on a person's breast; one or more green-light lasers on the interior of the cup which emit light energy into the breast; one or more light energy receivers on the interior of the cup which receive the light energy after it has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In another example, a wearable optical breast imaging device can be embodied in: a cup which is worn on a person's breast; one or more light emitters on the cup which emit near-infrared light into the breast, wherein different emitters in the array emit near-infrared light energy into the breast at different times; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a bra which is worn on a person's breasts; one or more light energy emitters on the (base) perimeter of a bra cup which emit light energy into a breast; and one or more light energy emitters on the (base) perimeter of the bra cup which receive the light energy after it has been transmitted through breast tissue, wherein the light energy is analyzed to create images of one or both of the breasts, identify the molecular and/or cellular composition of one or both of the breasts, and/or identify the locations and/or shapes of selected types of tissue within one or both of the breasts. Alternatively, a wearable optical breast imaging device can comprise: a cup which is configured to be worn on a person's breast, wherein the cup further comprises a convex elastic fabric portion which covers at least part of the front of the breast; one or more light energy emitters on the convex elastic fabric portion of the cup which are configured to emit light energy into the breast; and one or more light energy receivers on the convex elastic fabric portion of the cup which receive the light energy after the light energy has been transmitted through breast tissue; wherein light energy which has been transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; one or more light energy emitters on the cup which emit light energy into the breast; and one or more light energy receivers on the cup which receive light energy after it has been transmitted through breast tissue, wherein the transmitted light energy is analyzed using near-infrared spectroscopy in order to evaluate treatment effectiveness. In another example, a wearable optical breast imaging device can be embodied in: a cup which is worn on a person's breast; one or more light energy emitters on the cup which emit light energy into the breast; and one or more light energy receivers on the cup, wherein the light energy receivers receive the light energy after it has been transmitted through breast tissue, and wherein the light energy is analyzed to create an two-dimensional or three-dimensional image of the breast. A wearable optical breast imaging device can also comprise: a bra which is worn on a person's breasts; one or more light energy emitters on the interior of a bra cup which emit light energy into a breast; one or more light energy emitters on the interior of the bra cup which receive the light energy after it has been transmitted through breast tissue, wherein the light energy is analyzed to create images of one or both of the breasts, identify the molecular and/or cellular composition of one or both of the breasts, and/or identify the locations and/or shapes of selected types of tissue within one or both of the breasts; and a data processor and data transmitter on the back strap of the bra. Alternatively, a wearable optical breast imaging device can comprise: a cup which is worn on a person's breast; one or more near-infrared light energy emitters on the cup which emit light energy into the breast; and one or more light energy receivers on the cup which receive light energy after it has been transmitted through breast tissue, wherein the transmitted light energy is analyzed using near-infrared spectroscopy in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; one or more time-domain light emitters on the cup which emit short pulses of light energy into the breast; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can be embodied in: a bra which is worn on a person's breasts; wherein the bra further comprises two cups; and wherein each cup further comprises an array of light energy emitters around the (base) perimeter of the cup and an array of light energy receivers around the (base) perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a bra which is worn on a person's breasts; wherein the bra further comprises two cups; and wherein each cup further comprises an array of light energy emitters around the (base) perimeter of the cup and a light energy receiver on the perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a bra which is worn on a person's breasts; wherein the bra further comprises two cups; and wherein each cup further comprises an array of light energy emitters around the (base) perimeter of the cup and a light energy receiver on the lower perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; one or more light energy emitters on the arcuate upper perimeter and/or on the arcuate lower perimeter which emit light energy into the breast; and one or more light energy receivers on the arcuate upper perimeter and/or on the arcuate lower perimeter which receive the light energy transmitted through breast tissue; wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate lower perimeter which are activated sequentially to emit light energy into the breast at different times; and a plurality of light energy receivers on the arcuate upper perimeter which receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is configured to be worn on a person's breast; wherein the cup further comprises a convex elastic portion which covers at least part of the front of the breast; one or more light energy emitters on the cup which emit light energy into the breast; and one or more light energy receivers on the cup which receive the light energy after the light energy has been transmitted through breast tissue; wherein light energy which has been transmitted through breast tissue is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is configured to be worn on a person's breast; wherein the cup further comprises a convex elastic portion which covers at least part of the front of the breast; wherein the cup further comprises an arcuate upper perimeter which is configured to span an upper portion of the posterior base of the breast, wherein the arcuate upper perimeter comprises an arcuate member (such as a strap, wire, fold, or cuff) which is less elastic, less flexible, and/or more dense than the convex elastic portion; wherein the cup further comprises an arcuate lower perimeter which is configured to span a lower portion of the posterior base of the breast, wherein the arcuate lower perimeter comprises an arcuate member (such as a strap, wire, fold, or cuff) which is less elastic, less flexible, and/or more dense than the convex elastic portion; one or more light energy emitters on the arcuate lower perimeter of the cup which emit light energy into the breast; and one or more light energy receivers on the arcuate lower perimeter of the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast.

For example, a wearable optical breast imaging device can comprise: a cup which is worn on a person's breast; wherein the cup further comprises an arcuate lower (base) perimeter which spans a lower portion of the breast; one or more light energy emitters and one or more light energy receivers on the arcuate lower (base) perimeter, wherein the one or more light energy emitters emit light energy into the breast, wherein the one or more light energy receivers receive the light energy after it has been transmitted through breast tissue; wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; wherein the cup further comprises an arcuate upper perimeter which spans an upper portion of the breast; wherein the cup further comprises an arcuate lower perimeter which spans a lower portion of the breast; one or more light energy emitters on the arcuate lower perimeter which emit light energy into the breast; and one or more light energy receivers on the arcuate upper perimeter which receive the light energy after it has been transmitted through breast tissue; wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a concentric and/or nested ring array of light energy emitters on the interior of the cup and an array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a substantially-opaque cup which is worn on a person's breast; a concentric and/or nested ring array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a conic-section-shaped array of light energy emitters around a first portion of the perimeter of the cup and a conic-section-shaped array of light energy receivers around a second portion of the perimeter of the cup, wherein the first portion is on the opposite side of the breast from the second portion, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a conic-section-shaped array of light energy emitters on the perimeter of the cup and a conic-section-shaped array of light energy receivers on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a conic-section-shaped array of light energy emitters which are distributed along a first side of the perimeter of the cup and an array of light energy receivers which are distributed along a second side of the perimeter of the cup, wherein the second side is diametrically-opposite the first side, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a conic-section-shaped array of light energy emitters which are distributed along a first portion the perimeter of the cup, wherein the light energy emitters emit light energy into the breast, and wherein the first portion spans between the 10 o'clock to 2 o'clock positions (along the top of the cup); and an array of light energy receivers which are distributed along a second portion of the cup, wherein the light energy receivers receive the light energy after it has been transmitted through breast tissue, wherein the first portion spans between the 4 o'clock to 8 o'clock positions (along the bottom of the cup), and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; a convex array of light energy emitters and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a convex array of light energy emitters on the interior of the cup and a convex array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a convex array of light energy emitters on the perimeter of the cup which emit light energy into the breast from different locations at different times, wherein light emitters in the array are activated to emit light in a (counter) clockwise sequential manner; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a convex array of light energy emitters which spans at least half of the interior of the cup and which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of silicone-based optical transmission pathways which spans at least half of the interior of the cup and which emit light energy into the breast from different locations at different times; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a convex polar coordinate (e.g. spoke and ring) array of light energy emitters on the interior of the cup which emit light energy into the breast from different polar coordinate (e.g. spoke and ring) locations at different times; and one or more light energy receivers on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a downward-opening-convex array of light energy emitters on the interior of the cup; and an upward-opening-convex array of light energy receivers on the interior of the cup, wherein the light energy emitters emit light energy into the breast from different locations at different times, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the (base) perimeter of the cup which emits light into the breast with a wavelength in the range of 650 to 750 nm; a second light energy emitter on the (base) perimeter of the cup which emits light into the breast with a wavelength in the range of 750 nm to 850 nm; a third light energy emitter on the (base) perimeter of the cup which emits light into the breast with a wavelength in the range of 850 nm to 950 nm; and one or more light energy receivers on the (base) perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the cup which emits light into the breast with a first modulated wavelength at a first time; a second light energy emitter on the cup which emits light into the breast with a second modulated wavelength at a second time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; a first light energy emitter on the interior of the cup which emits light into the breast with a wavelength in the range of 650 to 700 nm; a second light energy emitter on the interior of the cup which emits light into the breast with a wavelength in the range of 700 nm to 750 nm; a third light energy emitter on the interior of the cup which emits light into the breast with a wavelength in the range of 750 nm to 800 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a first light energy emitter on the interior of the cup which emits light into the breast with a wavelength in the range of 600 to 700 nm; a second light energy emitter on the interior of the cup which emits light into the breast with a wavelength in the range of 700 nm to 800 nm; a third light energy emitter on the interior of the cup which emits light into the breast with a wavelength in the range of 800 nm to 900 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a first light energy emitter on the interior of the cup which emits light into the breast with a wavelength in the range of 600 to 800 nm at a first time; a second light energy emitter on the interior of the cup which emits light into the breast with a wavelength in the range of 800 nm to 1000 nm at a first time; a third light energy emitter on the interior of the cup which emits light into the breast with a wavelength in the range of 1000 nm to 1200 nm at a second time; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the interior of the cup which emits light into the breast at a first wavelength; a second light energy emitter on the interior of the cup which emits light into the breast at a second wavelength; and a light energy receiver on the interior of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a first light energy emitter on the perimeter of the cup which emits light into the breast with a wavelength in the range of 600 to 900 nm; a second light energy emitter on the perimeter of the cup which emits light into the breast with a wavelength in the range of 900 nm to 1200 nm; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the perimeter of the cup which emits light into the breast with a wavelength in the range of 600 to 900 nm; a second light energy emitter on the perimeter of the cup which emits light into the breast with a wavelength in the range of 900 nm to 1200 nm; a third light energy emitter on the perimeter of the cup which emits light into the breast with a wavelength in the range of 1200 nm to 1500 nm; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the perimeter of the cup which emits light into the breast with a wavelength in the range of 650 to 750 nm at a first time; a second light energy emitter on the perimeter of the cup which emits light into the breast with a wavelength in the range of 750 nm to 850 nm at a second time; a third light energy emitter on the perimeter of the cup which emits light into the breast with a wavelength in the range of 850 nm to 950 nm at a third time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the perimeter of the cup which emits light into the breast from a first location at a first wavelength at a first time; a second light energy emitter on the perimeter of the cup which emits light into the breast from a second location at a second wavelength at a second time; and a light energy receiver on the perimeter of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a helical array of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a hub-and-spoke array of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a light energy emitter on a first quadrant of the perimeter of the cup, wherein the light energy emitter emits light energy into the breast; and a light energy receiver on a second quadrant of the perimeter of the cup, wherein the second quadrant is diametrically-opposite the first quadrant, wherein the light energy receiver receives the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a multi-layer cup which is worn on a person's breast; a light energy emitter; a moving mirror, prism, or lens which is in optical communication with the light energy emitter; a radial-spoke array of flexible fiber optic pathways between interior and exterior layers of the multi-layer cup; wherein different flexible fiber optic pathways are in optical communication with the moving mirror, prism, or lens at different times as the mirror, prism, or lens moves; and wherein different flexible fiber optic pathways transmit light energy from the light energy emitter into the breast from different locations on the interior of the multi-layer cup at different times; and one or more light energy receivers on the multi-layer cup which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup with an inner layer and an outer layer which is worn on a person's breast; a light energy emitter; a plurality of silicone-based optical pathways between the inner layer and the outer layer which guide light from the light energy emitter to different locations on the cup, from which locations the light energy is transmitted into the person's breast; and a light energy receiver which receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a light energy emitter; an array of PDMS-based optical pathways which channel light from the light energy emitter to different locations around the (base) perimeter of the cup from which the light is transmitted into the breast at different times; and a light energy receiver on the perimeter of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a light energy receiver on the lower perimeter of the cup, and a radial-spoke array of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a light energy receiver on the lower perimeter of the cup, and an array of light energy emitters on the lower perimeter of the cup on either side of the light energy receiver, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a moving micromirror, prism, or lens; a plurality of flexible optical pathways spanning a portion of the cup which are in optical communication with the moving micromirror, prism, or lens; a light energy emitter, wherein light energy from the light energy emitter is guided to be transmitted into the breast from different positions on the interior of the cup at different times by the moving micromirror, prism, or lens and the plurality of flexible optical pathways; and a light energy receiver; wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a nested array of light energy emitters on the interior of the cup, wherein different light energy emitters in the array emit light energy into the breast at different times; and a light energy receiver on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a plurality of light energy emitters on an upper portion (e.g. the upper half) of the cup, wherein the plurality of light energy emitters emit light energy into the breast sequentially (at different locations at different times); and one or more light energy receivers on a lower portion (e.g. the lower half) of the cup, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a plurality of light energy emitters on the cup which emit light energy into the breast at different frequencies in the range of 600 nm to 1,000 nm; and one or more light energy emitters on the cup which receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a plurality of light energy emitters on the cup which emit light energy into the breast, wherein different light energy emitters in the array emit light energy into the breast at different wavelengths and at different times; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a plurality of light energy receivers on the lower perimeter of the cup, and a plurality of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a plurality of rings of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a radial-spoke array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a substantially-opaque cup which is worn on a person's breast; a radial-spoke ring array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a red-light laser on the perimeter of cup which emits light energy into the breast; a green-light laser on the perimeter of cup which emits light energy into the breast; and one or more light energy receivers on the perimeter of the cup which receive light energy from the red-light and green-light lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can comprise: a cup which is worn on a person's breast; a ring of light energy emitters and light energy receivers around the entire perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a ring of light energy emitters on the interior of the cup which emit light energy into the breast from different locations at different times, wherein light emitters in the array are activated to emit light in a (counter) clockwise sequential manner; and one or more light energy receivers on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a ring of light energy emitters on the interior of the cup; and a ring of light energy receivers on the interior of the cup, wherein the light energy emitters sequentially emit light energy into the breast, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; a semi-circular, semi-elliptical, or other conic section array of light energy emitters on the upper perimeter of the cup and a semi-circular, semi-elliptical, or other conic section array of light energy receivers on the lower perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a spoke-and-ring array of fiber optic light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an alternating sequence of light energy emitters and light energy receivers around at least half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an arcuate array of light energy emitters and an arcuate array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an arcuate array of light energy emitters on the interior of the cup and a light energy receiver on the interior of the cup, wherein different light energy emitters in the array emit light energy into the breast at different wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; an array light energy emitters distributed around the (base) perimeter of the cup, wherein the light energy emitters sequentially emit light energy into the breast; and a light energy receiver on the base perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of at least fifty light energy emitters on the (base) perimeter of the cup which emit light energy into the breast from different locations at different times; and an array of at least fifty light energy receivers on the (base) perimeter of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of at least fifty light energy emitters on the interior of the cup which emit light energy into the breast from different locations at different times; and an array of at least fifty light energy receivers on the interior of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of at least ten light energy emitters on the interior of the cup which emit light energy into the breast from different locations at different times; and a light energy receiver on the interior of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of at least ten light energy emitters on the upper half of the cup which emit light energy into the breast from different locations at different times; and an array of at least ten light energy receivers on the lower half of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: an elastic and/or stretchable fabric cup which is worn on a person's breast; an array of elastic light energy pathways which are part of the cup and transmit light energy into the breast from different locations on the interior of the cup; and a light energy receiver on the lower perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of fiber optic light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of fiber optic light energy emitters on the interior of the cup, wherein light energy emitters in the array emit light energy into the breast at different times, and a light energy receiver on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; an array of frequency-domain light emitters on the (base) perimeter of the cup which emit intensity-modulated light energy into the breast; and an array of light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the light energy is analyzed to identify levels of one or more substances selected from the group consisting of: collagen; hemoglobin, deoxyhemoglobin, and/or oxyhemoglobin; lipids; oxygen saturation; and water. In another embodiment, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the light energy is analyzed to identify one or more structures selected from the group consisting of: abnormal extracellular matrix; abnormal vasculature and/or vascular sprouting; and abnormal lymphatics.

For example, a wearable optical breast imaging device can be embodied in: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the phase of the light energy transmitted through breast tissue are analyzed to identify abnormal levels of one or more substances selected from the group consisting of: collagen; hemoglobin, deoxyhemoglobin, and/or oxyhemoglobin; lipids; oxygen saturation; and water. Alternatively, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the phase of the light energy transmitted through breast tissue are analyzed to create a map of the levels of one or more substances selected from the group consisting of: collagen; hemoglobin, deoxyhemoglobin, and/or oxyhemoglobin; lipids; oxygen saturation; and water.

A wearable optical breast imaging device can also comprise: a cup which is worn on a person's breast; an array of light energy emitters and light energy receivers which are distributed along the lower perimeter of the cup between the 3 o'clock and 9 o'clock locations on the perimeter, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters and light energy receivers which are evenly distributed around the (base) perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of light energy emitters and light energy receivers which are evenly distributed over the convex interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array of light energy emitters around at least a portion of the perimeter of the cup at different distances from a light energy receiver on the perimeter of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters on a first half (e.g. upper or lower) of the interior of the cup, wherein the light energy emitters sequentially emit light energy into the breast; and an array of light energy receivers on a second half (e.g. lower or upper) of the interior of the cup, wherein the first half is diametrically-opposite the second half, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of light energy emitters on a first side (e.g. right or left) of the interior of the cup, wherein the light energy emitters sequentially emit light energy into the breast; and an array of light energy receivers on a second side (e.g. left or right) of the interior of the cup, wherein the first half is diametrically-opposite the second half, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters on the cup which emit light energy into the breast from different locations at different times; and one or more light energy receivers on the cup which receive light energy after it has been transmitted through breast tissue, wherein the transmitted light energy is analyzed in order to create an image (or map) of breast tissue density. Alternatively, a wearable optical breast imaging device can comprise: an cup with an opaque layer which is worn on a person's breast; an array of light energy emitters on the cup, interior relative to the opaque layer; and a light energy receiver on the interior of the cup, interior relative to the opaque layer, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters on the interior of the cup and a light energy receiver on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters on the perimeter of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup with an opaque layer which is worn on a person's breast; an array of light energy emitters on the perimeter of the cup; and an array of light energy receivers on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an array of light energy emitters which collectively span at least 50% of the interior surface area of the cup and which emit light energy into the breast from different locations at different times; one or more light energy emitters on the interior of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can be embodied in: a cup which is worn on a person's breast; an array of light energy emitters which spans at least one-quarter of the perimeter of the cup and which emits light energy into the breast; one or more light energy receivers on the perimeter of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In an alternative example, a wearable optical breast imaging device can comprise: a cup which is worn on a person's breast; an array of matched pairs of light energy emitters and light energy receivers which is distributed around between 25% and 50% of the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of matched pairs of light energy emitters and light energy receivers on the interior of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 1 mm and 1 cm apart, and wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of matched pairs of light energy emitters and light energy receivers on the (base) perimeter of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 1 mm and 1 cm apart, and wherein light energy transmitted through breast tissue is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of matched pairs of light energy emitters and light energy receivers which is distributed around at least 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is diametrically-opposite the light energy emitter in the matched pair; and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of matched pairs of light energy emitters and light energy receivers which is distributed around between 25% and 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is in same quadrant of the cup as the light energy emitter in the matched pair; and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an array of matched pairs of light energy emitters and light energy receivers which is distributed around between 45% and 75% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is adjacent to the light energy emitter in the matched pair; and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of pairs of light energy emitters and light energy receivers around the (base) perimeter of the cup, wherein the a light energy receiver in a pair is diametrically-opposite a light energy emitter in the pair, wherein the light energy emitter in a pair emits light energy into the breast, wherein the light energy receiver in the pair receives the light energy after it has been transmitted through breast tissue, wherein different pairs are activated to emit and receive light at different wavelengths and at different times, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of pairs of light energy emitters and light energy receivers on the interior of the cup, wherein a light energy emitter in a pair emits light energy into the breast and a light energy receiver in the pair receives the light energy after it has been transmitted through breast tissue, wherein pairs of light energy emitters and receivers are activated sequentially, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: an elastic and/or stretchable fabric cup which is worn on a person's breast; an array of PDMS-based light energy pathways which are part of the cup and transmit light energy into the breast from different locations on the interior of the cup; and a light energy receiver on the perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of rings of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: an elastic and/or stretchable fabric cup which is worn on a person's breast; an array of silicone-based light energy pathways which are part of the cup and transmit light energy into the breast from different locations on the interior of the cup; and an array of light energy receivers in the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of time-domain light emitters on the cup which emit short pulses of light energy into the breast; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an array of triads, wherein each triad comprises two light energy emitters and one light energy receiver, and wherein the array of triads is distributed around at least 50% of the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an downwardly-convex array of light energy emitters on the interior of the cup and an upwardly-convex array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an upper arcuate array of light energy emitters on the upper interior surface of the cup which emit light into an upper portion of the breast; a lower arcuate array of light energy emitters on the lower interior surface of the cup which emit light into a lower portion of the breast; and an arcuate array of light energy receivers on the interior of the cup which receive light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an upper arcuate array of light energy emitters on the upper perimeter of the cup which emit light into an upper portion of the breast; a lower arcuate array of light energy emitters on the lower perimeter of the cup which emit light into a lower portion of the breast; and a light energy receiver on the perimeter of the cup which receives light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; at least one light energy emitter on a first portion of the perimeter of the cup which emits light into the breast; and at least one light energy receiver on a second portion of the perimeter of the cup which receives the light energy after it has been transmitted through breast tissue, wherein the second portion is diametrically-opposite the first portion, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; at least one light energy emitter on the interior of the cup which emits light into the breast; and at least one light energy receiver on the perimeter of the cup which receives the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; multiple rings of light energy emitters and light energy receivers around the interior convex surface of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; one or more first lasers on the interior of the cup which emit light energy into the breast with a wavelength in the range of 600 to 700 nm; one or more second lasers on the interior of the cup which emit light energy into the breast with a wavelength in the range of 850 to 950 nm; and one or more light energy emitters on the interior of the cup which receive light energy from the lasers after it has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can comprise: a cup which is worn on a person's breast; one or more frequency-domain light emitters on the (base) perimeter of the cup which emit intensity-modulated light energy into the breast; and one or more light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; one or more infra-red light emitters on the (base) perimeter of the cup which emit light energy into the breast; and one or more light receivers on the (base) perimeter of the cup which receive the light energy after it has been transmitted through breast tissue, and wherein the light energy transmitted through breast tissue is analyzed using frequency domain photon migration and broadband near-infrared spectroscopy. In another embodiment, a wearable optical breast imaging device can comprise: a cup which is worn on a person's breast; one or more light energy emitters and one or more light energy receivers on the interior of the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a bra which is worn on a person's breasts; one or more light energy emitters on the (base) perimeter of a bra cup which emit light energy into a breast; one or more light energy emitters on the (base) perimeter of the bra cup which receive the light energy after it has been transmitted through breast tissue, wherein the light energy is analyzed to create images of one or both of the breasts, identify the molecular and/or cellular composition of one or both of the breasts, and/or identify the locations and/or shapes of selected types of tissue within one or both of the breasts; and a data processor and data transmitter on the back strap of the bra. Alternatively, a wearable optical breast imaging device can comprise: a cup which is worn on a person's breast, wherein the cup further comprises a convex elastic fabric portion which covers at least part of the front of a breast and a cup base perimeter which is less elastic than the convex elastic fabric portion; one or more light energy emitters on the cup base perimeter which emit light energy into the breast; and one or more light energy receivers on the cup base perimeter which receive light energy after it has been transmitted through breast tissue; wherein light energy which has been transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; one or more light energy emitters on the cup which emit light energy into the breast; and one or more light energy receivers on the cup which receive light energy after it has been transmitted through breast tissue, wherein the transmitted light energy is analyzed using near-infrared spectroscopy in order to evaluate the effectiveness of medical treatment of abnormalities. In another example, a wearable optical breast imaging device can be embodied in: a cup which is worn on a person's breast; one or more light energy emitters on the cup which emit light with a modulated and/or variable wavelength into the breast; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; one or more light energy emitters on the interior of the cup which emit light energy into the breast; and one or more light energy emitters on the interior of the cup which receive light energy from the light energy emitters after it has been transmitted through breast tissue; wherein the light energy receivers are selected from the group consisting of photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor; and wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can comprise: a cup which is worn on a person's breast; one or more red-light lasers on the interior of the cup which emit light energy into the breast; one or more green-light lasers on the interior of the cup which emit light energy into the breast; and one or more light energy emitters on the interior of the cup which receive light energy from the red-light and green-light lasers after it has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

A wearable optical breast imaging device can also comprise: a cup which is worn on a person's breast; one or more time-domain light emitters on the interior of the cup which emit short pulses of light energy into the breast; and one or more light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a bra which is worn on a person's breasts; wherein the bra further comprises two cups; and wherein each cup further comprises an array of light energy emitters around at least half of the perimeter of the cup and an array of light energy receivers around at least half of the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a bra which is worn on a person's breasts; wherein the bra further comprises two cups; and wherein each cup further comprises an array of light energy emitters around at least half of the perimeter of the cup and a light energy receiver on the perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a bra which is worn on a person's breasts; wherein the bra further comprises two cups; and wherein each cup further comprises an array of light energy emitters around at least half of the perimeter of the cup and a light energy receiver on the lower perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate upper perimeter and/or on the arcuate lower perimeter which are activated sequentially to emit light energy into the breast at different times; and a light energy receiver on the arcuate upper perimeter and/or on the arcuate lower perimeter which is configured to receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate upper perimeter which are activated sequentially to emit light energy into the breast at different times; and a light energy receiver on the arcuate lower perimeter which is configured to receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast.

A wearable optical breast imaging device can also comprise: a cup which is configured to be worn on a person's breast; wherein the cup further comprises a convex elastic portion which covers at least part of the front of the breast; wherein the cup further comprises an arcuate upper perimeter which is configured to span an upper portion of the posterior base of the breast, wherein the arcuate upper perimeter comprises an arcuate member (such as a strap, wire, fold, or cuff) which is less elastic, less flexible, and/or more dense than the convex elastic portion; wherein the cup further comprises an arcuate lower perimeter which is configured to span a lower portion of the posterior base of the breast, wherein the arcuate lower perimeter comprises an arcuate member (such as a strap, wire, fold, or cuff) which is less elastic, less flexible, and/or more dense than the convex elastic portion; one or more light energy emitters on the cup which emit light energy into the breast; and one or more light energy receivers on the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast. Alternatively, a wearable optical breast imaging device can comprise: a cup which is configured to be worn on a person's breast; wherein the cup further comprises a convex elastic portion which covers at least part of the front of the breast; wherein the cup further comprises an arcuate upper perimeter which is configured to span an upper portion of the posterior base of the breast, wherein the arcuate upper perimeter comprises an arcuate member (such as a strap, wire, fold, or cuff) which is less elastic, less flexible, and/or more dense than the convex elastic portion; wherein the cup further comprises an arcuate lower perimeter which is configured to span a lower portion of the posterior base of the breast, wherein the arcuate lower perimeter comprises an arcuate member (such as a strap, wire, fold, or cuff) which is less elastic, less flexible, and/or more dense than the convex elastic portion; one or more light energy emitters on the arcuate upper perimeter of the cup which emit light energy into the breast; and one or more light energy receivers on the arcuate upper perimeter of the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast.

In an example, a wearable optical breast imaging device can comprise: a cup which is worn on a person's breast, wherein the cup further comprises a convex elastic fabric portion which covers at least part of the front of the breast; wherein the cup further comprises an arcuate upper perimeter which spans an upper portion of the breast, wherein the arcuate upper perimeter comprises an arcuate member (such as a strap, wire, fold, or cuff) which is less elastic, less flexible, and/or more dense than the convex elastic fabric portion; wherein the cup further comprises an arcuate lower perimeter which spans a lower portion of the breast, wherein the arcuate lower perimeter comprises an arcuate member (such as a strap, wire, fold, or cuff) which is less elastic, less flexible, and/or more dense than the convex elastic fabric portion; one or more light energy emitters on the arcuate upper perimeter and/or on the arcuate lower perimeter which emit light energy into the breast; and one or more light energy receivers on the arcuate upper perimeter and/or on the arcuate lower perimeter which receive the light energy after it has been transmitted through breast tissue; wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a concentric and/or nested ring array of light energy emitters on the interior of the cup and a light energy receiver on the interior of the cup, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can be embodied in: a substantially-opaque cup which is worn on a person's breast; a concentric and/or nested ring array of light energy emitters on the interior of the cup and one or more light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a conic-section-shaped array of light energy emitters around a first portion of the perimeter of the cup and a conic-section-shaped array of light energy receivers around a second portion of the perimeter of the cup, wherein the first portion is diametrically-opposite the breast from the second portion, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a conic-section-shaped array of light energy emitters on the perimeter of the cup and a light energy receiver on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a conic-section-shaped array of light energy emitters which are distributed along a first side of the perimeter of the cup and an array of light energy receivers which are distributed along a second side of the perimeter of the cup, wherein the second side is diametrically-opposite the first side, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of fiber optic light energy emitters on the interior of the cup and a light energy receiver on the interior of the cup, wherein the light energy emitters emit light energy into the breast from different locations at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a convex array of light energy emitters and a light energy receiver on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of light energy emitters on the interior of the cup which emit light energy into the breast from different locations at different times, wherein light emitters in the array are activated to emit light in a (counter) clockwise sequential manner; and one or more light energy receivers on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a convex array of light energy emitters on the upper perimeter of the cup and a convex array of light energy receivers on the lower perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a convex array of light energy emitters which spans at least three-quarters of the interior of the cup and which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a convex array of silicone-based optical transmission pathways which spans at least three-quarters of the interior of the cup and which emit light energy into the breast from different locations at different times; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex polar coordinate (e.g. spoke and ring) array of light energy emitters on the interior of the cup which emit light energy into the breast from different polar coordinate (e.g. spoke and ring) locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a downward-opening-convex array of light energy emitters on the interior of the cup; and an upward-opening-convex array of light energy receivers on the interior of the cup, wherein the light energy emitters sequentially emit light energy into the breast from different locations at different times, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; a first light energy emitter on the cup which emits coherent light in a first wavelength into the breast constantly during operation of the device; a second light energy emitter on the cup which emits coherent light in a second wavelength into the breast intermittently during operation of the device; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the cup which emits light into the breast constantly during operation of the device; a second light energy emitter on the cup which emits light into the breast in pulses during operation of the device; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the interior of the cup which emits light into the breast with a wavelength in the range of 650 to 750 nm; a second light energy emitter on the interior of the cup which emits light into the breast with a wavelength in the range of 750 nm to 850 nm; a third light energy emitter on the interior of the cup which emits light into the breast with a wavelength in the range of 850 nm to 950 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can be embodied in: a cup which is worn on a person's breast; a first light energy emitter on the interior of the cup which emits light into the breast with a wavelength in the range of 650 to 750 nm; a second light energy emitter on the interior of the cup which emits light into the breast with a wavelength in the range of 750 nm to 850 nm; a third light energy emitter on the interior of the cup which emits light into the breast with a wavelength in the range of 850 nm to 950 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the interior of the cup which emits light into the breast with a wavelength in the range of 600 to 700 nm at a first time; a second light energy emitter on the interior of the cup which emits light into the breast with a wavelength in the range of 700 nm to 800 nm at a second time; a third light energy emitter on the interior of the cup which emits light into the breast with a wavelength in the range of 800 nm to 900 nm at a third time; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a first light energy emitter on the interior of the cup which emits light into the breast at a first time; a second light energy emitter on the interior of the cup which emits light into the breast at a second time; and a light energy receiver on the interior of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the perimeter of the cup which emits light into the breast with a wavelength in the range of 600 to 800 nm; a second light energy emitter on the perimeter of the cup which emits light into the breast with a wavelength in the range of 800 nm to 1000 nm; a third light energy emitter on the perimeter of the cup which emits light into the breast with a wavelength in the range of 1000 nm to 1200 nm; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a first light energy emitter on the perimeter of the cup which emits light into the breast with a wavelength in the range of 600 to 900 nm at a first time; a second light energy emitter on the perimeter of the cup which emits light into the breast with a wavelength in the range of 900 nm to 1200 nm at a second time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the perimeter of the cup which emits light into the breast with a wavelength in the range of 600 to 900 nm at a first time; a second light energy emitter on the perimeter of the cup which emits light into the breast with a wavelength in the range of 900 nm to 1200 nm at a second time; a third light energy emitter on the perimeter of the cup which emits light into the breast with a wavelength in the range of 1200 nm to 1500 nm at a third time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a first light energy emitter on the perimeter of the cup which emits a continuous beam of light into the breast from a first location at a first wavelength at a first time; a second light energy emitter on the perimeter of the cup which emits short pulses of light into the breast from a second location at a second wavelength at a second time; and a light energy receiver on the perimeter of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a helical array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; a hub-and-spoke array of light energy emitters on the interior of the cup, wherein spokes with multiple light energy emitters are activated to emit light energy into the breast in a sequential radially rotating manner (like radar around a central hub); one or more light energy receivers which receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a light energy emitter; a moving mirror, prism, or lens which is in optical communication with the light energy emitter; a radial-spoke array of flexible fiber optic pathways which is integrated into the cup; wherein different flexible fiber optic pathways are in optical communication with the moving mirror, prism, or lens at different times as the mirror, prism, or lens moves; and wherein different flexible fiber optic pathways transmit light energy from the light energy emitter into the breast from different locations on the interior of the cup at different times; and one or more light energy receivers on the cup which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: an cup which is worn on a person's breast; a light energy emitter; a moving mirror, prism, or lens which is in optical communication with the light energy emitter; a plurality of flexible fiber optic pathways in the cup; wherein different flexible fiber optic pathways are in optical communication with the moving mirror, prism, or lens at different times as the mirror, prism, or lens moves; and wherein different flexible fiber optic pathways transmit light energy from the light energy emitter into the breast from different locations on the interior of the cup at different times; and one or more light energy receivers on the cup which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a light energy emitter; an array of optical (e.g. fiber optic) pathways in the cup which channel light from the light energy emitter to different locations on the interior of the cup from which the light is transmitted into the breast at different times; and a light energy receiver on the interior of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a light energy emitter; an array of silicone-based optical pathways in the cup which channel light from the light energy emitter to different locations on the interior of the cup from which the light is transmitted into the breast at different times; and a light energy receiver on the interior of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a light energy receiver on the lower perimeter of the cup, and a ring of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a light energy receiver on the lower perimeter of the cup, and an array of light energy emitters on the upper perimeter of the cup, wherein at least one of the light energy emitters is diametrically-opposite the light energy receiver, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a moving micromirror, prism, or lens; a plurality of silicone-based optical pathways spanning a portion of the cup which are in optical communication with the moving micromirror, prism, or lens; a light energy emitter, wherein light energy from the light energy emitter is guided to be transmitted into the breast from different positions on the interior of the cup at different times by the moving micromirror, prism, or lens and the plurality of flexible optical pathways; and a light energy receiver; wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a nested-ring array of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is configured to be worn on a person's breast, wherein the cup further comprises a convex elastic fabric portion which covers at least part of the front of the breast; a plurality of light energy emitters on the convex elastic fabric portion which are activated sequentially to emit light energy into the breast at different times; and a light energy receiver on the convex elastic fabric portion which receives the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a plurality of light energy emitters on the cup which emit light energy into the breast, wherein different light energy emitters in the array emit light energy into the breast at different wavelengths; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a plurality of light energy emitters on the cup, wherein the plurality of light energy emitters emit light energy into the breast sequentially (at different locations at different times); and one or more light energy receivers on the cup, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: an elastic fabric cup which is worn on a person's breast; a plurality of more flexible fiber optic light energy emitters which are woven or sewn into the elastic fabric cup; and one or more light energy receivers on the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a polar coordinate (e.g. spoke and ring) array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; a radial-spoke array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a substantially-opaque cup which is worn on a person's breast; a radial-spoke ring array of light energy emitters on the interior of the cup and one or more light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a red-light laser on the perimeter of cup which emits light energy into the breast; an infrared laser on the perimeter of cup which emits light energy into the breast; and one or more light energy receivers on the perimeter of the cup which receive light energy from the red-light and infrared lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

For example, a wearable optical breast imaging device can be embodied in: a cup which is worn on a person's breast; a ring of light energy emitters and light energy receivers on the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a ring of light energy emitters on the interior of the cup, wherein different light energy emitters in the array emit light energy into the breast at different times; and a light energy receiver on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a ring of light energy emitters on the perimeter of the cup which emit light energy into the breast from different locations at different times, wherein light emitters in the array are activated to emit light in a (counter) clockwise sequential manner; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a sequential array which alternates between light energy emitters and light energy receivers around a portion of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; a spoke-and-ring array of fiber optic light energy emitters on the interior of the cup and a light energy receiver on the interior of the cup, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an arcuate array of light energy emitters along the bottom of the cup and an arcuate array of light energy receivers along the top of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an arcuate array of light energy emitters and an arcuate array of light energy receivers on the interior of the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an arcuate array of light energy emitters on the interior of the cup and a light energy receiver on the interior of the cup, wherein different light energy emitters in the array emit light energy into the breast at varying and/or modulated wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array light energy emitters distributed around the (base) perimeter of the cup, wherein the light energy emitters sequentially emit light energy into the breast; and a light energy receiver on the perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of at least fifty light energy emitters on the interior of the cup which emit light energy into the breast from different locations at different times; and a light energy receiver on the (base) perimeter of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of at least ten light energy emitters on the (base) perimeter of the cup which emit light energy into the breast from different locations at different times; and a light energy receiver on the (base) perimeter of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of at least ten light energy emitters on the interior of the cup which emit light energy into the breast from different locations at different times; and an array of at least ten light energy receivers on the (base) perimeter of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of continuous wave light emitters on the (base) perimeter of the cup which emit light energy into the breast; and an array of light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: an elastic and/or stretchable fabric cup which is worn on a person's breast; an array of elastic light energy pathways which are part of the cup and transmit light energy into the breast from different locations on the interior of the cup; and a light energy receiver on the perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of fiber optic light energy emitters on the interior of the cup and a light energy receiver on the interior of the cup, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; an array of fiber optic light energy emitters on the interior of the cup, wherein light energy emitters in the array emit light energy into the breast at different times, and an array of light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of frequency-domain light emitters on the cup which emit intensity-modulated light energy into the breast; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the spectrum of the light energy are analyzed to identify levels of one or more substances selected from the group consisting of: collagen; hemoglobin, deoxyhemoglobin, and/or oxyhemoglobin; lipids; oxygen saturation; and water.

In an example, a wearable optical breast imaging device can be embodied in: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the light energy is analyzed to identify abnormal levels of one or more substances selected from the group consisting of: collagen; hemoglobin, deoxyhemoglobin, and/or oxyhemoglobin; lipids; oxygen saturation; and water. In another embodiment, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the light energy is analyzed to create a map of the levels of one or more substances selected from the group consisting of: collagen; hemoglobin, deoxyhemoglobin, and/or oxyhemoglobin; lipids; oxygen saturation; and water.

In an example, a wearable optical breast imaging device can be embodied in: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein light energy transmitted through breast tissue is analyzed using spectroscopy to identify the levels of one or more substances selected from the group consisting of: collagen; hemoglobin, deoxyhemoglobin, and/or oxyhemoglobin; lipids; oxygen saturation; and water. Alternatively, a wearable optical breast imaging device can comprise: a cup which is worn on a person's breast; an array of light energy emitters and light energy receivers which are distributed along the lower perimeter of the cup spanning the 4 o'clock and 8 o'clock locations on the perimeter, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of light energy emitters and light energy receivers which are evenly distributed around half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters and light energy receivers which are evenly distributed around the convex interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an array of light energy emitters around at least one-half of the base perimeter of the cup, wherein the light energy emitters sequentially emit light energy into the breast; and an array of light energy receivers around at least one-half of the base perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of light energy emitters on a first half (upper or lower) of the base perimeter of the cup, wherein the light energy emitters sequentially emit light energy into the breast; and an array of light energy receivers on a second half (lower or upper) of the base perimeter of the cup, wherein the first half is diametrically-opposite the second half, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of light energy emitters on a first side (right or left) of the interior of the cup, wherein the light energy emitters sequentially emit light energy into the breast; and an array of light energy receivers on a second side (left or right) of the interior of the cup, wherein the second side is diametrically-opposite the first side, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters on the cup which emits light energy into the breast; one or more light energy receivers on the perimeter of the cup which receive light energy transmitted through breast tissue, wherein light transmitted through breast tissue is analyzed using diffuse optical imaging.

In an example, a wearable optical breast imaging device can comprise: an elastic and/or stretchable fabric cup with an opaque layer which is worn on a person's breast; an array of light energy emitters on the cup, interior relative to the opaque layer; and a light energy receiver on the interior of the cup, interior relative to the opaque layer, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of light energy emitters on the interior of the cup and a light energy receiver on the lower perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: an elastic and/or stretchable fabric cup which is worn on a person's breast; an array of light energy emitters on the perimeter of the cup and an array of light energy receivers on the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters which are distributed across the interior of the cup, wherein the light energy emitters emit light energy into the breast; and a light energy receiver on the interior of the cup, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an array of light energy emitters which collectively span at least 75% of the interior surface area of the cup and which emit light energy into the breast from different locations at different times; one or more light energy emitters on the interior of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In another embodiment, a wearable optical breast imaging device can comprise: a cup which is worn on a person's breast; an array of light-transmitting pathways on the cup, wherein different light-transmitting pathways sequentially emit light energy into the breast at different locations at different times; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of matched pairs of light energy emitters and light energy receivers which is distributed around between 25% and 50% of the (base) perimeter of the cup, wherein a light energy emitter in a matched pair emits light energy into the breast and a light energy receiver in the matched pair receives receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; an array of matched pairs of light energy emitters and light energy receivers on the interior of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 0.5 cm and 1 cm apart, and wherein light energy transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; an array of matched pairs of light energy emitters and light energy receivers on the (base) perimeter of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 0.5 cm and 1 cm apart, and wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of matched pairs of light energy emitters and light energy receivers which is distributed around at least 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is in same quadrant of the cup as the light energy emitter in the matched pair; and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of matched pairs of light energy emitters and light energy receivers which is distributed around between 25% and 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is adjacent to the light energy emitter in the matched pair; and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of nested rings of light energy emitters on the interior of the cup, wherein different light energy emitters in the array emit light energy into the breast at different times; and a light energy receiver on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; an array of pairs of light energy emitters and light energy receivers around the (base) perimeter of the cup, wherein the a light energy receiver in a pair is adjacent to a light energy emitter in the pair, wherein the light energy emitter in a pair emits light energy into the breast, wherein the light energy receiver in the pair receives the light energy after it has been transmitted through breast tissue, wherein different pairs are activated to emit and receive light at different wavelengths and at different times, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of pairs of light energy emitters and light energy receivers on the interior of the cup, wherein a light energy emitter in a pair emits light energy into the breast and a light energy receiver in the pair receives the light energy after it has been transmitted through breast tissue, wherein pairs of light energy emitters and receivers are separated by between 1 cm and 3 cm, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: an elastic and/or stretchable fabric cup which is worn on a person's breast; an array of PDMS-based light energy pathways which are part of the cup and transmit light energy into the breast from different locations on the interior of the cup; and an array of light energy receivers in the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of rings of light energy emitters on the interior of the cup, wherein different light energy emitters in the array emit light energy into the breast at different times; and a light energy receiver on the interior of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: an elastic and/or stretchable fabric cup which is worn on a person's breast; an array of silicone-based light energy pathways which are part of the cup and transmit light energy into the breast from different locations on the interior of the cup; and an array of light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of time-domain light emitters on the interior of the cup which emit short pulses of light energy into the breast; and an array of light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of triads, wherein each triad comprises two light energy emitters on either side of a light energy receiver, and wherein the array of triads is distributed around at least 50% of the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; an ultraviolet laser on the interior of the cup which emits light energy into the breast; an infrared laser on the interior of the cup which emits light energy into the breast; and one or more light energy receivers on the interior of the cup which receive light energy from the ultraviolet and infrared lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

For example, a wearable optical breast imaging device can be embodied in: a cup which is worn on a person's breast; an upper arcuate array of light energy emitters on the upper interior surface of the cup which emit light into an upper portion of the breast; a lower arcuate array of light energy emitters on the lower interior surface of the cup which emit light into a lower portion of the breast; and a light energy receiver on the interior of the cup which receives light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an upper perimeter of the cup which comprises an arcuate hem, strap, fold, wire, or cuff; a lower perimeter of the cup which comprises an arcuate hem, strap, fold, wire, or cuff; one or more light energy emitters on the upper perimeter which emit light energy into the breast; and one or more light energy receivers on the lower perimeter which receive light energy transmitted through breast tissue, wherein broadening of the laser pulses due to transmission through the breast is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can comprise: a cup which is worn on a person's breast; at least one light energy emitter on a first portion of the perimeter of the cup which emits light into the breast; and at least one light energy receiver on a second portion of the perimeter of the cup which receives the light energy after it has been transmitted through breast tissue, wherein the second portion is in the same quadrant of the perimeter as the first portion, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; at least one light energy emitter on the perimeter of the cup which emits light into the breast; and at least one light energy receiver on the interior of the cup which receives the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can be embodied in: a cup which is worn on a person's breast; one or more continuous wave light emitters on the (base) perimeter of the cup which emit light energy into the breast; and one or more light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; one or more flexible fiber optic light energy emitters and one or more light energy receivers on the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is worn on a person's breast; one or more frequency-domain light emitters on the cup which emit intensity-modulated light energy into the breast; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; one or more infra-red light emitters on the cup which emit light energy into the breast; and one or more light receivers on the cup which receive the light energy after it has been transmitted through breast tissue, and wherein the light energy transmitted through breast tissue is analyzed using frequency domain photon migration and broadband near-infrared spectroscopy.

In an example, a wearable optical breast imaging device can be embodied in: a cup which is worn on a person's breast; one or more light energy emitters and one or more light energy receivers on the interior of the cup, wherein the one or more light energy emitters emit light energy into the breast, wherein the one or more light energy receivers receive the light energy after it has been transmitted through breast tissue, and wherein absorption, scattering, and/or transmission of light energy through the breast is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; one or more light energy emitters on the (base) perimeter of the cup which emit light energy into the breast; and one or more light energy emitters on the (base) perimeter of the cup which receive light energy from the light energy emitters after it has been transmitted through breast tissue; wherein the light energy receivers are selected from the group consisting of photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor; and wherein the light energy is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can comprise: a cup which is worn on a person's breast; one or more light energy emitters on the cup which emit light energy into the breast; and one or more light energy emitters on the cup which receive light energy from the light energy emitters after it has been transmitted through breast tissue; wherein the light energy receivers are selected from the group consisting of photodetector, photoresistor, avalanche photodiode (APD), charge-coupled device (CCD), complementary metal-oxide semiconductor (CMOS), infrared detector, infrared photoconductor, infrared photodiode, light dependent resistor (LDR), optoelectric sensor, photoconductor, photodiode, photomultiplier, and phototransistor; and wherein the light energy is analyzed to create an image of the breast, identify the molecular and/or cellular composition of the breast, and/or identify the locations and/or shapes of selected types of tissue within the breast; and a data processor and data transmitter. In another embodiment, a wearable optical breast imaging device can comprise: a cup which is worn on a person's breast; one or more light energy emitters on the cup which emit light energy into the breast, wherein the light energy emitters are selected from the group consisting of light emitting diode (LED), coherent light source, organic light emitting diode (OLED), laser, laser diode, infrared light-energy emitter, multi-wavelength source, resonant cavity light emitting diode (RCLED), super-luminescent light emitting diode (SLED), and ultraviolet light-energy emitter; and one or more light energy receivers on the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

For example, a wearable optical breast imaging device can be embodied in: a cup which is worn on a person's breast; one or more light energy emitters on the cup which emit light with a modulated and/or variable intensity into the breast; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; one or more light energy emitters on the interior of the cup which emit light energy into the breast, wherein the light energy emitters are selected from the group consisting of light emitting diode (LED), coherent light source, organic light emitting diode (OLED), laser, laser diode, infrared light-energy emitter, multi-wavelength source, resonant cavity light emitting diode (RCLED), super-luminescent light emitting diode (SLED), and ultraviolet light-energy emitter; and one or more light energy receivers on the interior of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can be embodied in: a cup which is worn on a person's breast; one or more red-light lasers on the interior of the cup which emit light energy into the breast; one or more light energy receivers on the interior of the cup which receive the light energy after it has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In another example, a wearable optical breast imaging device can comprise: a bra with cups which are worn on a person's breasts; wherein a cup further comprises a convex elastic fabric portion which covers at least part of the front of a breast; one or more light energy emitters on the convex elastic fabric portion of the cup which are configured to emit light energy into the breast; and one or more light energy receivers on the convex elastic fabric portion of the cup which are configured to receive the light energy after the light energy has been transmitted through breast tissue; wherein light energy which has been transmitted through breast tissue is analyzed in order to create an image of the breast, identify the molecular and/or cellular composition of the breast, and/or identify the locations and/or shapes of selected types of tissue within the breast.

For example, a wearable optical breast imaging device can comprise: a bra which is worn on a person's breasts; wherein the bra further comprises two cups; and wherein each cup further comprises an array of light energy emitters around at least one-quarter of the perimeter of the cup and an array of light energy receivers around at least one-quarter of the perimeter of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a bra which is worn on a person's breasts; wherein the bra further comprises two cups; and wherein each cup further comprises an array of light energy emitters around at least one-quarter of the perimeter of the cup and a light energy receiver on the perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a bra which is worn on a person's breasts; wherein the bra further comprises two cups; and wherein each cup further comprises an array of light energy emitters around at least one-quarter of the perimeter of the cup and a light energy receiver on the lower perimeter of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate upper perimeter and/or on the arcuate lower perimeter which are activated sequentially to emit light energy into the breast at different times; and a plurality of light energy receivers on the arcuate upper perimeter and/or on the arcuate lower perimeter which receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate lower perimeter which are activated sequentially to emit light energy into the breast at different times; and a light energy receiver on the arcuate upper perimeter which is configured to receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is configured to be worn on a person's breast; wherein the cup further comprises a convex elastic portion which covers at least part of the front of the breast; wherein the cup further comprises an arcuate upper perimeter which is configured to span an upper portion of the posterior base of the breast, wherein the arcuate upper perimeter comprises an arcuate member (such as a strap, wire, fold, or cuff) which is less elastic, less flexible, and/or more dense than the convex elastic portion; wherein the cup further comprises an arcuate lower perimeter which is configured to span a lower portion of the posterior base of the breast, wherein the arcuate lower perimeter comprises an arcuate member (such as a strap, wire, fold, or cuff) which is less elastic, less flexible, and/or more dense than the convex elastic portion; one or more light energy emitters on the arcuate upper perimeter of the cup which emit light energy into the breast; and one or more light energy receivers on the arcuate lower perimeter of the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; wherein the cup further comprises a fiber optic array through which light energy is emitted into the breast from different locations; and one or more light energy emitters on the cup which receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; wherein the cup further comprises an arcuate upper perimeter which spans an upper portion of the breast; wherein the cup further comprises an arcuate lower perimeter which spans a lower portion of the breast; one or more light energy emitters on the arcuate upper perimeter and/or on the arcuate lower perimeter which emit light energy into the breast; and one or more light energy receivers on the arcuate upper perimeter and/or on the arcuate lower perimeter which receive the light energy after it has been transmitted through breast tissue; wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a concentric and/or nested ring array of light energy emitters on the interior of the cup and a light energy receiver on the interior of the cup, wherein different light energy emitters in the array emit light energy into the breast at different wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a conic-section-shaped array of light energy emitters around a first portion of the perimeter of the cup and a conic-section-shaped array of light energy receivers around a second portion of the perimeter of the cup, wherein the first portion is above the second portion, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a conic-section-shaped array of light energy emitters on the interior of the cup and a conic-section-shaped array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a conic-section-shaped array of light energy emitters which are distributed along a first portion the perimeter of the cup and an array of light energy receivers which are distributed along a second portion of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a conic-section-shaped array of light energy emitters which are distributed along a first side of the perimeter of the cup and an array of light energy receivers which are distributed along a second side of the perimeter of the cup, wherein the second side is diametrically-opposite the first side, wherein different light energy emitters in the array emit light energy into the breast at different wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a convex array of fiber optic light energy emitters on the interior of the cup and a light energy receiver on the interior of the cup, wherein different light energy emitters in the array emit light energy into the breast at different times, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a convex array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a convex array of light energy emitters on the interior of the cup which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array of light energy emitters on the upper perimeter of the cup and a concave array of light energy receivers on the lower perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a convex array of optical transmission pathways which spans at least half of the interior of the cup and which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a convex array which alternates between light energy emitters and light energy receivers around at least half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a convex polar coordinate (e.g. spoke and ring) array of light energy emitters on the interior of the cup which emit light energy into the breast from different polar coordinate (e.g. spoke and ring) locations at different times; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a downward-opening-convex array of light energy emitters on the interior of the cup; and one or more light energy receivers on the interior of the cup, wherein the light energy emitters sequentially emit light energy into the breast from different locations at different times, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the cup which emits light in a first wavelength into the breast constantly during operation of the device; a second light energy emitter on the cup which emits light in a second wavelength into the breast intermittently during operation of the device; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the cup which emits light into the breast constantly during operation of the device; a second light energy emitter on the cup which emits light into the breast intermittently during operation of the device; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a first light energy emitter on the interior of the cup which emits light into the breast with a wavelength in the range of 600 to 900 nm; a second light energy emitter on the interior of the cup which emits light into the breast with a wavelength in the range of 900 nm to 1200 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the interior of the cup which emits light into the breast with a wavelength in the range of 600 to 900 nm; a second light energy emitter on the interior of the cup which emits light into the breast with a wavelength in the range of 900 nm to 1200 nm; a third light energy emitter on the interior of the cup which emits light into the breast with a wavelength in the range of 1200 nm to 1500 nm; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the interior of the cup which emits light into the breast with a wavelength in the range of 650 to 750 nm at a first time; a second light energy emitter on the interior of the cup which emits light into the breast with a wavelength in the range of 750 nm to 850 nm at a second time; a third light energy emitter on the interior of the cup which emits light into the breast with a wavelength in the range of 850 nm to 950 nm at a third time; and one or more light energy receivers on the interior of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a first light energy emitter on the interior of the cup which emits light into the breast from a first location at a first wavelength at a first time; a second light energy emitter on the interior of the cup which emits light into the breast from a second location at a second wavelength at a second time; and a light energy receiver on the interior of the cup, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a first light energy emitter on the perimeter of the cup which emits light into the breast with a wavelength in the range of 600 to 700 nm; a second light energy emitter on the perimeter of the cup which emits light into the breast with a wavelength in the range of 700 nm to 800 nm; a third light energy emitter on the perimeter of the cup which emits light into the breast with a wavelength in the range of 800 nm to 900 nm; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a first light energy emitter on the perimeter of the cup which emits light into the breast with a wavelength in the range of 600 to 800 nm at a first time; a second light energy emitter on the perimeter of the cup which emits light into the breast with a wavelength in the range of 800 nm to 1000 nm at a second time; a third light energy emitter on the perimeter of the cup which emits light into the breast with a wavelength in the range of 1000 nm to 1200 nm at a third time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a first light energy emitter on the perimeter of the cup which emits light into the breast with a first wavelength at a first time; a second light energy emitter on the perimeter of the cup which emits light into the breast with a second wavelength at a second time; and one or more light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a first light energy emitter on the perimeter of the cup which emits light into the breast at a first time; a second light energy emitter on the perimeter of the cup which emits light into the breast at a second time; and a light energy receiver on the perimeter of the cup between the first light emitter and the second light emitter, wherein the light energy receivers receive light energy after the light energy has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a helical array of light energy emitters on the interior of the cup which emit light energy into the breast from different locations at different times; and one or more light energy receivers on the lower perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a latitude-and-longitude array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can be embodied in: a fabric cup which is worn on a person's breast; a light energy emitter; a moving mirror, prism, or lens which is in optical communication with the light energy emitter; a plurality of flexible fiber optic pathways which are sewn or woven into the fabric of the cup; wherein different flexible fiber optic pathways are in optical communication with the moving mirror, prism, or lens at different times as the mirror, prism, or lens moves; and wherein different flexible fiber optic pathways transmit light energy from the light energy emitter into the breast from different locations on the interior of the cup at different times; and one or more light energy receivers on the cup which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup with an inner layer and an outer layer which is worn on a person's breast; a light energy emitter; a plurality of flexible optical pathways between the inner layer and the outer layer which guide light from the light energy emitter to different locations on the cup, from which locations the light energy is transmitted into the person's breast; and a light energy receiver which receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a light energy emitter; an array of optical (e.g. fiber optic) pathways which channel light from the light energy emitter to different locations around the (base) perimeter of the cup from which the light is transmitted into the breast at different times; and a light energy receiver on the perimeter of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a light energy emitter; an array of silicone-based optical pathways which channel light from the light energy emitter to different locations around the (base) perimeter of the cup from which the light is transmitted into the breast at different times; and a light energy receiver on the perimeter of the cup, wherein the light energy detector receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a light energy receiver on the lower perimeter of the cup, and a spoke-and-ring array of light energy emitters on convex interior surface of the cup, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; a light energy receiver on the perimeter of the cup, and an array of light energy emitters on the perimeter of the cup on either side of the light energy receiver, wherein different light energy emitters emit light energy into the breast at different times, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; a moving micromirror, prism, or lens; a radial-spoke array of optical pathways spanning a portion of the cup which are in optical communication with the moving micromirror, prism, or lens; a light energy emitter, wherein light energy from the light energy emitter is guided to be transmitted into the breast from different positions on the interior of the cup at different times by the moving micromirror, prism, or lens and the plurality of flexible optical pathways; and a light energy receiver; wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a plurality of fiber optic light energy emitters and one or more light energy receivers on the cup, wherein the plurality of light energy emitters emit light energy into the breast sequentially (at different locations at different times), wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is configured to be worn on a person's breast, wherein the cup further comprises a convex elastic fabric portion which covers at least part of the front of the breast; a plurality of light energy emitters on the convex elastic fabric portion which are activated sequentially to emit light energy into the breast at different times; and a plurality of light energy receivers on the convex elastic fabric portion which receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a plurality of light energy emitters on the cup which emit light energy into the breast, wherein different light energy emitters in the array emit light energy into the breast at different wavelengths at the same time; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a plurality of light energy emitters on the upper half of cup, wherein the plurality of light energy emitters emit light energy into the breast sequentially (at different locations at different times); and one or more light energy receivers on the lower half of the cup, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is configured to be worn on a person's breast, wherein the cup further comprises a convex elastic fabric portion which covers at least part of the front of the breast; a plurality of pairs of light energy emitters and light energy receivers on the convex elastic fabric portion, wherein light energy emitters are activated sequentially to emit light energy into the breast at different times, wherein light energy receivers receive the light energy after it has been transmitted through breast tissue, and wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a polar-coordinate array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; a radial-spoke ring array of light energy emitters on the interior of the cup and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can comprise: a cup which is worn on a person's breast; a red-light laser on the interior of the cup which emits light energy into the breast; a green-light laser on the interior of the cup which emits light energy into the breast; and one or more light energy receivers on the interior of the cup which receive light energy from the red-light and green-light lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can comprise: a cup which is worn on a person's breast; a ring of light energy emitters and light energy receivers around the (base) perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; a ring of light energy emitters and light energy receivers which are equidistantly distributed around the (base) perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; a ring of light energy emitters on the interior of the cup; and a light energy receiver on the interior of the cup, wherein the light energy emitters sequentially emit light energy into the breast, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; a semi-circular, semi-elliptical, or other conic section array of light energy emitters and light energy receivers on the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; a sequential array which alternates between light energy emitters and light energy receivers around at least half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; a spoke-and-ring array of light energy emitters and light energy receivers on the interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an arcuate array of light energy emitters along the top of the cup and an arcuate array of light energy receivers along the bottom of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can comprise: a cup which is worn on a person's breast; an arcuate array of light energy emitters around at least half of the perimeter of the cup which emit light energy into the breast from different locations at different times, wherein light emitters in the array are activated to emit light in a (counter) clockwise sequential manner; and one or more light energy receivers on the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an arcuate array of light energy emitters on the interior of the cup and a light energy receiver on the interior of the cup, wherein different light energy emitters in the array emit light energy into the breast at varying and/or modulated intensities, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array light energy emitters distributed around the upper perimeter of the cup, wherein the light energy emitters sequentially emit light energy into the breast; and a light energy receiver on the lower perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of at least fifty light energy emitters on the interior of the cup which emit light energy into the breast from different locations at different times; and a light energy receiver on the interior of the cup which receives light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; an array of at least ten light energy emitters on the (base) perimeter of the cup which emit light energy into the breast from different locations at different times; and an array of at least ten light energy receivers on the (base) perimeter of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of at least ten light energy emitters on the interior of the cup which emit light energy into the breast from different locations at different times; and an array of at least ten light energy receivers on the interior of the cup which receive light energy after it has been transmitted through breast tissue, wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of continuous wave light emitters on the cup which emit light energy into the breast; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: an elastic and/or stretchable fabric cup which is worn on a person's breast; an array of elastic light energy pathways which are part of the cup and transmit light energy into the breast from different locations on the interior of the cup; and an array of light energy receivers in the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of fiber optic light energy emitters on the interior of the cup and a light energy receiver on the interior of the cup, wherein different light energy emitters in the array emit light energy into the breast at different wavelengths, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of fiber optic light energy emitters on the interior of the cup, wherein light energy emitters in the array emit light energy into the breast at different times, and an array of light energy receivers around at least half of the perimeter of the cup, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of frequency-domain light emitters on the interior of the cup which emit intensity-modulated light energy into the breast; and an array of light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the absorption, scattering, and/or transmission of the light energy through the breast are analyzed to identify levels of one or more substances selected from the group consisting of: collagen; hemoglobin, deoxyhemoglobin, and/or oxyhemoglobin; lipids; oxygen saturation; and water. In another example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the spectrum of the light energy are analyzed to identify abnormal levels of one or more substances selected from the group consisting of: collagen; hemoglobin, deoxyhemoglobin, and/or oxyhemoglobin; lipids; oxygen saturation; and water.

For example, a wearable optical breast imaging device can comprise: a cup which is worn on a person's breast; an array of light emitters on the cup which emit light energy into the breast, wherein different emitters in the array emit light energy into the breast at different times; and an array of light energy receivers on the cup, wherein the light energy receivers receive the light energy after it has been transmitted through breast tissue, and wherein the changes in the spectrum of the light energy are analyzed to create a map of the levels of one or more substances selected from the group consisting of: collagen; hemoglobin, deoxyhemoglobin, and/or oxyhemoglobin; lipids; oxygen saturation; and water. Alternatively, a wearable optical breast imaging device can be embodied in: a cup which is worn on a person's breast; an array of light emitters on the cup which emit short pulses of light energy into the breast, wherein different emitters in the array emit light short energy pulses into the breast at different times; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters and light energy receivers which are distributed along the lower perimeter of the cup spanning the 5 o'clock and 7 o'clock locations on the perimeter, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; an array of light energy emitters and light energy receivers which are evenly distributed around the lower half of the perimeter of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of light energy emitters and light energy receivers which are distributed in rings around the convex interior of the cup, wherein the light energy emitters emit light into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of light energy emitters around at least one-quarter of the base perimeter of the cup, wherein the light energy emitters sequentially emit light energy into the breast; and an array of light energy receivers around at least one-quarter of the base perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of light energy emitters on a first quadrant of the interior of the cup, wherein the light energy emitters sequentially emit light energy into the breast; and an array of light energy receivers on a second quadrant of the interior of the cup, wherein the first quadrant is diametrically-opposite the second quadrant, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can be embodied in: a cup which is worn on a person's breast; an array of light energy emitters on a first side (right or left) of the base perimeter of the cup, wherein the light energy emitters sequentially emit light energy into the breast; and an array of light energy receivers on a second side (left or right) of the base perimeter of the cup, wherein the second side is diametrically-opposite the first side, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of light energy emitters on the cup which emits light energy into the breast; one or more light energy receivers on the perimeter of the cup which receive light energy transmitted through breast tissue, wherein light transmitted through breast tissue is analyzed using (broadband) spectroscopy. Alternatively, a wearable optical breast imaging device can be embodied in: an cup with an opaque layer which is worn on a person's breast; an array of light energy emitters on the cup, interior relative to the opaque layer; and an array of light energy receivers on the interior of the cup, interior relative to the opaque layer, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup with an opaque layer which is worn on a person's breast; an array of light energy emitters on the interior of the cup; and a light energy receiver on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of light energy emitters on the perimeter of the cup which spans at least half of the perimeter of the cup and which emits light energy into the breast; one or more light energy receivers on the perimeter of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

A wearable optical breast imaging device can also comprise: a cup which is worn on a person's breast; an array of light energy emitters which are evenly distributed around the (base) perimeter of the cup, wherein the light energy emitters sequentially emit light energy into the breast from different locations at different times; and an array of light energy receivers which are evenly distributed around the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; an array of light energy emitters which collectively span at least one-quarter of the interior surface of cup and which emit light energy into the breast; one or more light energy receivers on the interior of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter.

In an example, a wearable optical breast imaging device can comprise: a cup which is worn on a person's breast; an array of matched pairs of light energy emitters and light energy receivers which is distributed around at least 50% of the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; an array of matched pairs of light energy emitters and light energy receivers which is distributed around between 45% and 75% of the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of matched pairs of light energy emitters and light energy receivers on the interior of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 0.5 cm and 1.5 cm apart, and wherein light energy transmitted through breast tissue is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array of matched pairs of light energy emitters and light energy receivers on the (base) perimeter of the cup, wherein a light energy emitter in a matched pair emits light energy into the breast and a light energy receiver in the matched pair receives the light energy after it has been transmitted through breast tissue, wherein a light energy emitter and a light energy receiver in a matched pair are between 0.5 cm and 1.5 cm apart, and wherein light energy transmitted through breast tissue is analyzed in order to create an image of the breast, identify the molecular and/or cellular composition of the breast, and/or identify the locations and/or shapes of selected types of tissue within the breast.

A wearable optical breast imaging device can also comprise: a cup which is worn on a person's breast; an array of matched pairs of light energy emitters and light energy receivers which is distributed around at least 50% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is adjacent to the light energy emitter in the matched pair; and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; an array of matched pairs of light energy emitters and light energy receivers which is distributed around between 45% and 75% of the (base) perimeter of the cup, wherein a light energy emitter in a matched pair emits light energy into the breast and a light energy in the matched pair receives receive the light energy after it has been transmitted through breast tissue, wherein the light energy receiver in a matched pair is diametrically-opposite the light energy emitter in the matched pair; and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; an array of pairs of light energy emitters and light energy receivers around the (base) perimeter of the cup, wherein the a light energy receiver in a pair is diametrically-opposite a light energy emitter in the pair, wherein the light energy emitter in a pair emits light energy into the breast, wherein the light energy receiver in the pair receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is worn on a person's breast; an array of pairs of light energy emitters and light energy receivers distributed around the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of pairs of light energy emitters and light energy receivers on the interior of the cup, wherein a light energy emitter in a pair emits light energy into the breast and a light energy receiver in the pair receives the light energy after it has been transmitted through breast tissue, wherein pairs of light energy emitters and receivers are separated by between 1 cm and 5 cm, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: an elastic and/or stretchable fabric cup which is worn on a person's breast; an array of PDMS-based light energy pathways which are part of the cup and transmit light energy into the breast from different locations on the interior of the cup; and an array of light energy receivers on the perimeter of the cup, wherein the light energy receivers receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

For example, a wearable optical breast imaging device can comprise: an elastic and/or stretchable fabric cup which is worn on a person's breast; an array of silicone-based light energy pathways which are part of the cup and transmit light energy into the breast from different locations on the interior of the cup; and a light energy receiver on the lower perimeter of the cup, wherein the light energy receiver receives light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can be embodied in: a cup which is worn on a person's breast; an array of silicone-based light-transmitting pathways on the cup, wherein different light-transmitting pathways sequentially emit light energy into the breast at different locations at different times; and an array of light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an array of triads, each comprising two light energy emitters and one light energy receiver, wherein the array is distributed around the (base) perimeter of the cup, wherein the light energy emitters sequentially emit light energy into the breast and the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In an alternative example, a wearable optical breast imaging device can be embodied in: a cup which is worn on a person's breast; an array of triads; wherein each triad comprises a first light energy emitter which emits light energy into the breast at a first wavelength, a second light energy emitter which emits light energy into the breast at a second wavelength, and a light energy receiver; wherein the array of triads is distributed around at least 50% of the (base) perimeter of the cup, wherein a light energy receiver receives light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an ultraviolet laser on the perimeter of cup which emits light energy into the breast; an infrared laser on the perimeter of cup which emits light energy into the breast; and one or more light energy receivers on the perimeter of the cup which receive light energy from the ultraviolet and infrared lasers after the light energy has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can comprise: a cup which is worn on a person's breast; an upper arcuate array of light energy emitters on the upper perimeter of the cup which emit light into an upper portion of the breast; a lower arcuate array of light energy emitters on the lower perimeter of the cup which emit light into a lower portion of the breast; an upper arcuate array of light energy receivers on the upper perimeter of the cup which receive light energy after it has been transmitted through breast tissue; and a lower arcuate array of light energy emitters on the lower perimeter of the cup which receive light energy after it has been transmitted through breast tissue, wherein light energy which has been transmitted through breast tissue is analyzed to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; an upper perimeter of the cup which comprises an arcuate hem, strap, fold, wire, or cuff; a lower perimeter of the cup which comprises an arcuate hem, strap, fold, wire, or cuff; one or more light energy emitters on the lower perimeter which emit light energy into the breast; and one or more light energy receivers on the upper perimeter which receive light energy transmitted through breast tissue, wherein broadening of the laser pulses due to transmission through the breast is analyzed to create an image of the breast, identify the molecular and/or cellular composition of the breast, and/or identify the locations and/or shapes of selected types of tissue within the breast; and a data processor and data transmitter. In another example, a wearable optical breast imaging device can be embodied in: a cup which is worn on a person's breast; at least one light energy emitter on a first portion of the perimeter of the cup which emits light into the breast; and at least one light energy receiver on a second portion of the perimeter of the cup which receives the light energy after it has been transmitted through breast tissue, wherein the second portion is in the opposite quadrant of the perimeter relative to the first portion, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; at least one light energy emitter on the perimeter of the cup which emits light into the breast; and at least one light energy receiver on the perimeter of the cup which receives the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can be embodied in: a cup which is worn on a person's breast; one or more continuous wave light emitters on the cup which emit light energy into the breast; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

A wearable optical breast imaging device can also comprise: a cup which is worn on a person's breast; one or more flexible fiber optic light energy emitters which are woven into the cup; and one or more light energy receivers on the cup, wherein the one or more light energy emitters emit light energy into the breast, wherein the one or more light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In another embodiment, a wearable optical breast imaging device can be embodied in: a cup which is worn on a person's breast; one or more frequency-domain light emitters on the interior of the cup which emit intensity-modulated light energy into the breast; and one or more light energy receivers on the interior of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

For example, a wearable optical breast imaging device can comprise: a cup which is worn on a person's breast; one or more light emitters on the cup which emit coherent light into the breast, wherein different emitters in the array emit coherent light energy into the breast at different times; and one or more light energy receivers on the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast. Alternatively, a wearable optical breast imaging device can comprise: a cup with an inner layer and an outer layer which is worn on a person's breast; one or more light energy emitters between the inner layer and the outer layer which emit light energy into the person's breast; and one of more light energy receivers between the inner layer and the outer layer which receive light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a cup which is worn on a person's breast; one or more light energy emitters on the (base) perimeter of the cup which emit light energy into the breast, wherein the light energy emitters are selected from the group consisting of light emitting diode (LED), coherent light source, organic light emitting diode (OLED), laser, laser diode, infrared light-energy emitter, multi-wavelength source, resonant cavity light emitting diode (RCLED), super-luminescent light emitting diode (SLED), and ultraviolet light-energy emitter; and one or more light energy receivers on the (base) perimeter of the cup which receive light energy transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. In another example, a wearable optical breast imaging device can be embodied in: a cup which is worn on a person's breast; one or more light energy emitters on the cup which emit light energy into the breast; and one or more light energy receivers on the cup which receive light energy after it has been transmitted through breast tissue, wherein the transmitted light energy is analyzed using long wavelength broadband spectroscopy in order to create an image of the breast, identify the molecular and/or cellular composition of the breast, and/or identify the locations and/or shapes of selected types of tissue within the breast.

For example, a wearable optical breast imaging device can comprise: a cup which is configured to be worn on a person's breast, wherein the cup further comprises a convex elastic fabric portion which covers at least part of the front of the breast; one or more light energy emitters on the cup which emit light energy into the breast; and one or more light energy receivers on the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast. A wearable optical breast imaging device can be embodied in: a bra which is worn on a person's breasts; one or more light energy emitters on the interior of a bra cup which emit light energy into a breast; and one or more light energy emitters on the interior of the bra cup which receive the light energy after it has been transmitted through breast tissue, wherein the light energy is analyzed to create images of one or both of the breasts, identify the molecular and/or cellular composition of one or both of the breasts, and/or identify the locations and/or shapes of selected types of tissue within one or both of the breasts.

A wearable optical breast imaging device can also comprise: a cup which is worn on a person's breast; one or more light energy emitters on the interior of the cup which emit light energy into the breast; one or more light energy receivers on the interior of the cup which receive the light energy after it has been transmitted through breast tissue, wherein the light energy is analyzed to create an image of the breast, identify the molecular and/or cellular composition of the breast, and/or identify the locations and/or shapes of selected types of tissue within the breast; and a data processor and data transmitter. Alternatively, a wearable optical breast imaging device can be embodied in: a cup which is worn on a person's breast; one or more time-domain light emitters on the (base) perimeter of the cup which emit short pulses of light energy into the breast; and one or more light energy receivers on the (base) perimeter of the cup, wherein the light energy receivers receive the light energy after it has been transmitted through breast tissue, and wherein the light energy is analyzed to create an image of the breast, identify the molecular and/or cellular composition of the breast, and/or identify the locations and/or shapes of selected types of tissue within the breast.

In an example, a wearable optical breast imaging device can comprise: a bra which is worn on a person's breasts; wherein the bra further comprises two cups; and wherein each cup further comprises an array of light energy emitters on the interior of the cup and an array of light energy receivers on the interior of the cup, wherein the light energy emitters emit light energy into the breast, wherein the light energy receivers receive the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. In an alternative example, a wearable optical breast imaging device can be embodied in: a bra which is worn on a person's breasts; wherein the bra further comprises two cups; and wherein each cup further comprises an array of light energy emitters on the interior of the cup and a light energy receiver on the interior of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast.

In an example, a wearable optical breast imaging device can comprise: a bra which is worn on a person's breasts; wherein the bra further comprises two cups; and wherein each cup further comprises an array of light energy emitters on the interior of the cup and a light energy receiver on the interior of the cup, wherein the light energy emitters emit light energy into the breast, wherein the light energy receiver receives the light energy after it has been transmitted through breast tissue, and wherein the transmitted light energy is analyzed in order to create an image of the breast, identify the molecular and/or cellular composition of the breast, and/or identify the locations and/or shapes of selected types of tissue within the breast. Alternatively, a wearable optical breast imaging device can comprise: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast, wherein the arcuate upper perimeter comprises an arcuate member (such as a strap, wire, fold, or cuff) which is less elastic, less flexible, and/or more dense than the convex elastic fabric portion; wherein the cup further comprises an arcuate lower perimeter which is configured to span a lower portion of the breast, wherein the arcuate lower perimeter comprises an arcuate member (such as a strap, wire, fold, or cuff) which is less elastic, less flexible, and/or more dense than the convex elastic fabric portion; one or more light energy emitters on the arcuate upper perimeter and/or on the arcuate lower perimeter which emit light energy into the breast; and one or more light energy receivers on the arcuate upper perimeter and/or on the arcuate lower perimeter which receive the light energy transmitted through breast tissue; wherein light energy transmitted through breast tissue is analyzed in order to create an image of the breast, identify the molecular and/or cellular composition of the breast, and/or identify the locations and/or shapes of selected types of tissue within the breast.

For example, a wearable optical breast imaging device can be embodied in: a cup which is configured to be worn on a person's breast; wherein the cup further comprises a convex elastic fabric portion which covers at least part of the front of the breast; wherein the cup further comprises an arcuate upper perimeter which is configured to span an upper portion of the breast; wherein the cup further comprises an arcuate lower perimeter which is configured to span the lower base of the breast; a plurality of light energy emitters on the arcuate upper perimeter which are activated sequentially to emit light energy into the breast at different times; and a plurality of light energy receivers on the arcuate lower perimeter which receive the light energy transmitted through breast tissue, wherein light energy transmitted through breast tissue is analyzed in order to create an image of the breast, identify the molecular and/or cellular composition of the breast, and/or identify the locations and/or shapes of selected types of tissue within the breast. In another example, a wearable optical breast imaging device can comprise: a cup which is configured to be worn on a person's breast; wherein the cup further comprises a convex elastic portion which covers at least part of the front of the breast; one or more light energy emitters on the convex elastic portion of the cup which emit light energy into the breast; and one or more light energy receivers on the convex elastic portion of the cup which receive the light energy after the light energy has been transmitted through breast tissue; wherein light energy which has been transmitted through breast tissue is analyzed in order to create an image of the breast, identify the molecular and/or cellular composition of the breast, and/or identify the locations and/or shapes of selected types of tissue within the breast.

In an example, a wearable optical breast imaging device can be embodied in: a cup which is configured to be worn on a person's breast; wherein the cup further comprises a convex elastic portion which covers at least part of the front of the breast; wherein the cup further comprises an arcuate upper perimeter which is configured to span an upper portion of the posterior base of the breast, wherein the arcuate upper perimeter comprises an arcuate member (such as a strap, wire, fold, or cuff) which is less elastic, less flexible, and/or more dense than the convex elastic portion; wherein the cup further comprises an arcuate lower perimeter which is configured to span a lower portion of the posterior base of the breast, wherein the arcuate lower perimeter comprises an arcuate member (such as a strap, wire, fold, or cuff) which is less elastic, less flexible, and/or more dense than the convex elastic portion; one or more light energy emitters on the arcuate lower perimeter of the cup which emit light energy into the breast; and one or more light energy receivers on the arcuate upper perimeter of the cup which receive the light energy after the light energy has been transmitted through breast tissue; wherein light energy which has been transmitted through breast tissue is analyzed in order to—create an image of the breast, identify the molecular and/or cellular composition of the breast, and/or identify the locations and/or shapes of selected types of tissue within the breast.

A wearable optical breast imaging device can also comprise: a cup which is worn on a person's breast; wherein the cup further comprises a fiber optic array through which light energy is emitted into the breast from different locations around the upper perimeter of the cup; and one or more light energy emitters on the lower perimeter of the cup which receive the light energy after it has been transmitted through breast tissue, and wherein the light energy is analyzed to create an image of the breast, identify the molecular and/or cellular composition of the breast, and/or identify the locations and/or shapes of selected types of tissue within the breast. In another example, a wearable optical breast imaging device can comprise: a cup which is worn on a person's breast; wherein the cup further comprises an arcuate upper perimeter which spans an upper portion of the breast; wherein the cup further comprises an arcuate lower perimeter which spans a lower portion of the breast; one or more light energy emitters on the arcuate upper perimeter which emit light energy into the breast; and one or more light energy receivers on the arcuate lower perimeter which receive the light energy after it has been transmitted through breast tissue; wherein the transmitted light energy is analyzed in order to create an image of the breast, identify the molecular and/or cellular composition of the breast, and/or identify the locations and/or shapes of selected types of tissue within the breast.

I claim:

1. A wearable optical breast imaging device comprising:
at least one cup which is configured to be worn on at least one breast;
an array of light emitters on the interior of the cup which emit light beams into the breast at different times; and
an array of light receivers on the interior of the cup which receive the light beams after they have been transmitted through breast tissue, wherein changes in the spectra of the light beams caused by transmission of the light beams through the breast tissue are analyzed in order to diagnose whether there is abnormal tissue within the breast and/or to identify the location of abnormal tissue within the breast.

2. A wearable optical breast imaging device comprising:
at least one cup which is configured to be worn on at least one breast;
wherein the cup further comprises a first light transmission pair comprising a first light emitter and a first light receiver and wherein the first light transmission pair transmits light through the breast tissue along a first vector at a first time;
wherein the cup further comprises a second light transmission pair comprising a second light emitter and a second light receiver and wherein the second light transmission pair transmits light through the breast tissue along a second vector at a second time;
wherein the cup further comprises a third light transmission pair comprising a third light emitter and a third light receiver and wherein the third light transmission pair transmits light through the breast tissue along a third vector at a third time;
wherein the three vectors intersect in three-dimensional space within the breast tissue; and
wherein changes in the spectra of the light beams from the three light transmission pairs caused by transmission of the light beams through the breast tissue are jointly analyzed in order to determine whether there is abnormal tissue at the location where the three vectors intersect.
